SEQUENCE LISTING (10 sequences)

SEQ ID NO: 1 - protein, 5 aa, Homo sapiens
EYGMH

SEQ ID NO: 2 - protein, 17 aa, Homo sapiens
VIGFAGLIKGYADSVKG

SEQ ID NO: 3 - protein, 15 aa, Homo sapiens
EGAGSNIYYYYGMDV

SEQ ID NO: 4 - protein, 124 aa, Homo sapiens
QVQLVESGGGVVQPGRSLRLSCAASGFTFSEYGMHWVRQAPGKGLEWVAVIGFAGLIKGYADSVKGRFTISRDNSKNTLYLQMNSLRAEDTAVYYCAREGAGSNIYYYYGMDVWGQGTTVTVSS

SEQ ID NO: 5 - protein, 454 aa, Homo sapiens
QVQLVESGGGVVQPGRSLRLSCAASGFTFSEYGMHWVRQAPGKGLEWVAVIGFAGLIKGYADSVKGRFTISRDNSKNTLYLQMNSLRAEDTAVYYCAREGAGSNIYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 11 aa, Homo sapiens
RASQSVSSSYL

SEQ ID NO: 7 - protein, 7 aa, Homo sapiens
GASSRAT

SEQ ID NO: 8 - protein, 10 aa, Homo sapiens
QQYGSSPIFT

SEQ ID NO: 9 - protein, 109 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPIFTFGPGTKVDIK

SEQ ID NO: 10 - protein, 216 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPIFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2021/0009632 A1
(43) Pub. Date: Jan. 14, 2021

(54) METHODS OF PURIFYING MONOMERIC MONOCLONAL ANTIBODIES

(71) Applicant: BRISTOL-MYERS SQUIBB COMPANY, Princeton, NJ (US)

(72) Inventors: Zhijun Tan, Acton, MA (US); Vivekh Ehamparanathan, Acton, MA (US); Yuanli Song, Acton, MA (US); Angela T. Lewandowski, Northborough, MA (US); Zhengjian Li, Sudbury, MA (US)

(21) Appl. No.: 17/042,984

(22) PCT Filed: Mar. 28, 2019
(86) PCT No.: PCT/US2019/024565

§ 371 Date: Sep. 29, 2020

Related U.S. Application Data
(60) Provisional application No. 62/649,976, filed on Mar. 29, 2018.

(51) Int. Cl. C07K 1/18; C07K 16/24; C07K 1/16; C07K 1/22; C12N 5/07

(52) U.S. Cl. CPC C07K 1/18; C07K 16/24; C07K 1/165; C12N 2500/30; C12N 5/06; C07K 2317/565; C12N 2500/60; C07K 1/22

(57) ABSTRACT

In certain embodiments, the present invention provides a method of purifying a monomeric monoclonal antibody from a mixture which comprises the monomeric monoclonal antibody and one or more contaminants, comprising: a) subjecting the mixture to cation exchange chromatography (CEX) matrix, wherein the monomeric monoclonal antibody binds to the CEX matrix; b) contacting the CEX matrix with a wash solution at a pH which is between about 7 and about 7.8; c) eluting the monomeric monoclonal antibody from the CEX matrix into an elution solution, thereby purifying the monomeric monoclonal antibody.

### CROSS REFERENCE TO RELATED APPLICATIONS

This application claims the benefit of U.S. Provisional Application No. 62/649,976, filed Mar. 29, 2018, the entirety of which is incorporated by reference herein.

### BACKGROUND OF THE INVENTION

The large-scale, economic purification of proteins is an increasingly important problem for the biopharmaceutical industry. Therapeutic proteins are typically produced using prokaryotic or eukaryotic cell lines that are engineered to express the protein of interest from a recombinant plasmid containing the gene encoding the protein. Separation of the desired protein from the mixture of components fed to the cells, cellular by-products, and aggregate forms of the protein, to an adequate purity, e.g., sufficient for use as a human therapeutic, poses a formidable challenge to biologics manufacturers.

Accordingly, there is a need in the art for alternative protein purification methods that can be used to expedite the large-scale processing of protein-based therapeutics, such as antibodies.

### SUMMARY OF THE INVENTION

In certain embodiments, the present invention provides a method of purifying a monomeric protein of interest, from a mixture which comprises the protein of interest and one or more contaminants.

In certain specific embodiments, the present invention provides a method of purifying a monomeric monoclonal antibody (e.g., an anti-IP10 monoclonal antibody) from a mixture which comprises the monomeric monoclonal antibody and one or more contaminants, comprising: a) subjecting the mixture to cation exchange chromatography (CEX) matrix, wherein the monomeric monoclonal antibody binds to the CEX matrix; b) contacting the CEX matrix with a wash solution at a pH which is between about 7 and about 7.8; c) eluting the monomeric monoclonal antibody from the CEX matrix into an elution solution, thereby purifying the monomeric monoclonal antibody. To illustrate, the contaminants are selected from aggregates of the monoclonal antibody, host cell proteins, host cell metabolites, host cell constitutive proteins, nucleic acids, endotoxins, viruses, product related contaminants, lipids, media additives and media derivatives. For example, aggregates of the anti-IP10 monoclonal antibody comprise dimers, multimers, and an intermediate aggregate species. Optionally, the intermediate aggregate species is removed in step (b).

In certain aspects, the mixture is selected from a harvested cell culture fluid, a cell culture supernatant, and a conditioned cell culture supernatant, a cell lysate, and a clarified bulk. For example, the cell culture is a mammalian cell culture, such as a Chinese Hamster Ovary (CHO) cell culture.

In certain aspects, the mixture of the present method has been obtained by an affinity chromatography (e.g., Protein A affinity chromatography). Optionally, the elution solution from the CEX step is not subjected to a second chromatography step. Optionally, the elution solution from the CEX step is further subjected to a second chromatography step, such as an ion exchange chromatography, a hydrophobic interaction chromatography, and a mix-mode chromatography.

In certain aspects, the pH of the wash solution is between about 7.2 and about 7.6 (e.g., 7.2, 7.3, 7.4, 7.5, 7.6). Optionally, the salt concentration of the wash buffer is between about 20 and 40 mM, such as between about 24 and 30 mM.

In certain aspects, the anti-IP10 monoclonal antibody comprises heavy chain CDR1, CDR2, and CDR3 amino acid sequences of SEQ ID NOs: 1, 2, and 3, respectively. In certain aspects, the anti-IP10 monoclonal antibody comprises light chain CDR1, CDR2, and CDR3 amino acid sequences of SEQ ID NOs: 6, 7, and 8, respectively. To illustrate, the anti-IP10 monoclonal antibody comprises heavy chain CDR1, CDR2, and CDR3 sequences of SEQ ID NOs: 1, 2, and 3, and light chain CDR1, CDR2, and CDR3 sequences of SEQ ID NOs: 6, 7, and 8, respectively. To illustrate, the anti-IP10 monoclonal antibody comprises a heavy variable region sequence and a light chain variable region sequence of SEQ ID NOs: 4 and 9, respectively. To illustrate, the anti-IP10 monoclonal antibody comprises the full-length heavy chain amino acid sequence and the full-length light chain amino acid sequence of SEQ ID NOs: 5 and 10, respectively.

In certain specific embodiments, the monomeric anti-IP10 monoclonal antibody is purified to at least 90% monomer purity, optionally at least 95% monomer purity, or optionally at least 99% monomer purity.

### BRIEF DESCRIPTION OF THE DRAWING

FIG. 1 shows the anti-IP10 mAb CEX salt gradient (0 mM to 300 mM NaCl in 50 mM Acetate, pH 5.5).

FIG. 2 shows the anti-IP10 mAb CEX: load condition (50 mM acetate, pH 5.5), elution condition (50 mM acetate, 100 mM NaCl, pH 5.5). The high order aggregate and dimer were successfully removed. However, the intermediate aggregate remained at the same level in the elution pool, indicating a co-elution between the intermediate and monomer.

FIG. 3 shows the anti-IP10 mAb SEC profile after MEP Hypercel chromatography.

FIG. 4 shows the intermediate species (dotted line) and the starting material (solid line) fractionated using a prep SEC column.

FIG. 5 shows overlay of the capillary electropheragrams for the intermediate species and monomers under non-reducing condition (A) and reducing condition (B).

FIG. 6 shows the intermediate species, monomers, and dimers on a WCX-10 HPLC column and HIC butyl column. A—WCX-10 column at running buffer condition pH 6.0, peaks from left to right: monomer, intermediate, dimer; B—WCX-10 column at running buffer condition pH 7.0, peaks from left to right: intermediate, monomer, dimer; C—HIC butyl column, peaks from left to right: intermediate, monomer, dimer.

FIG. 7 shows the iCE profile for the intermediate species vs monomers (Black line—monomers; Red line—intermediate species).

FIG. 8 shows ESI/MS Chromatograms.

FIG. 9 shows ESI/MS Chromatograms.

FIG. 10 shows the CEX pH Gradient using buffer A (40 mM phosphate, pH 5.5) and buffer B (35 mM phosphate, pH 8.5).

FIG. 11 shows species percentage versus fraction using pH gradient elution.

FIG. 12 shows the cumulative species vs overall cumulative mass using pH gradient and salt gradient, respectively.

FIG. 13 shows the pH Gradient (pH5.5→8.5) at various salt concentrations (20, 25, 30 and 35 mM phosphate).

FIG. 14 shows the cumulative intermediate species % vs cumulative mass under pH Gradient (pH5.5→8.5) at various salt concentrations (20, 25, 30 and 35 mM phosphate).

FIG. 15 shows size exclusion chromatograms of samples of load, wash, elution, and strip under the optimized CEX column condition.

FIG. 16 shows CEX DOE results by evaluating column load amount, wash pH, wash salt, and wash volume.

FIG. 17 shows Isotherm and partition coefficient.

### DETAILED DESCRIPTION OF THE INVENTION

The present invention provides a method of purifying a monomeric protein of interest, from a mixture which comprises the protein of interest and one or more contaminants.

In certain specific embodiments, the present invention provides a method of purifying a monomeric anti-IP10 monoclonal antibody from a mixture which comprises the monomeric anti-IP10 monoclonal antibody and one or more contaminants, comprising: a) subjecting the mixture to cation exchange chromatography (CEX) matrix, wherein the monomeric anti-IP10 monoclonal antibody binds to the CEX matrix; b) contacting the CEX matrix with a wash solution at a pH which is between about 7 and about 7.8; c) eluting the monomeric anti-IP10 monoclonal antibody from the CEX matrix into an elution solution, thereby purifying the monomeric anti-IP10 monoclonal antibody. To illustrate, the contaminants are selected from aggregates of the anti-IP10 monoclonal antibody, host cell proteins, host cell metabolites, host cell constitutive proteins, nucleic acids, endotoxins, viruses, product related contaminants, lipids, media additives and media derivatives. For example, aggregates of the anti-IP10 monoclonal antibody comprise dimers, multimers, and an intermediate aggregate species. Optionally, the intermediate aggregate species is removed in step (b).

In certain aspects, the mixture is selected from a harvested cell culture fluid, a cell culture supernatant, and a conditioned cell culture supernatant, a cell lysate, and a clarified bulk. For example, the cell culture is a mammalian cell culture, such as a Chinese Hamster Ovary (CHO) cell culture.

In certain aspects, the mixture of the present method has been obtained by an affinity chromatography (e.g., Protein A affinity chromatography). Optionally, the elution solution from the CEX step is not subjected to a second chromatography step.

Optionally, the elution solution from the CEX step is further subjected to a second chromatography step, such as an ion exchange chromatography, a hydrophobic interaction chromatography, and a mix-mode chromatography.

In certain aspects, the pH of the wash solution is between about 7.2 and about 7.6 (e.g., 7.2, 7.3, 7.4, 7.5, and 7.6). Optionally, the salt concentration of the wash buffer is between about 20 and 40 mM, such as between about 24 and 30 mM.

In certain aspects, the anti-IP10 monoclonal antibody comprises heavy chain CDR1, CDR2, and CDR3 amino acid sequences of SEQ ID NOs: 1, 2, and 3, respectively. In certain aspects, the anti-IP10 monoclonal antibody comprises light chain CDR1, CDR2, and CDR3 amino acid sequences of SEQ ID NOs: 6, 7, and 8, respectively. To illustrate, the anti-IP10 monoclonal antibody comprises heavy chain CDR1, CDR2, and CDR3 sequences of SEQ ID NOs: 1, 2, and 3, and light chain CDR1, CDR2, and CDR3 sequences of SEQ ID NOs: 6, 7, and 8, respectively. To illustrate, the anti-IP10 monoclonal antibody comprises a heavy variable region sequence and a light chain variable region sequence of SEQ ID NOs: 4 and 9, respectively. To illustrate, the anti-IP10 monoclonal antibody comprises the full-length heavy chain amino acid sequence and the full-length light chain amino acid sequence of SEQ ID NOs: 5 and 10, respectively.

In certain aspects, the monomeric anti-IP10 monoclonal antibody is purified to at least 90% monomer purity, optionally at least 95% monomer purity, or optionally at least 99% monomer purity.

### I. Definitions

In order that the present disclosure may be more readily understood, certain terms are first defined. As used in this application, except as otherwise expressly provided herein, each of the following terms shall have the meaning set forth below. Additional definitions are set forth throughout the application.

As used herein, the term “protein of interest” is used in its broadest sense to include any protein (either natural or recombinant), present in a mixture, for which purification is desired. Such proteins of interest include, without limitation, hormones, growth factors, cytokines, immunoglobulins (e.g., antibodies), and immunoglobulin-like domain-containing molecules (e.g., ankyrin or fibronectin domain-containing molecules).

As used herein, a “cell culture” refers to cells in a liquid medium. Optionally, the cell culture is contained in a bioreactor. The cells in a cell culture can be from any organism including, for example, bacteria, fungus, insects, mammals or plants. In a particular embodiment, the cells in a cell culture include cells transfected with an expression construct containing a nucleic acid that encodes a protein of interest (e.g., an antibody). Suitable liquid media include, for example, nutrient media and non-nutrient media. In a particular embodiment, the cell culture comprises a Chinese Hamster Ovary (CHO) cell line in nutrient media, not subject to purification by, for example, filtration or centrifugation.

As used herein, the term “clarified bulk” refers to a mixture from which particulate matter has been substantially removed. Clarified bulk includes cell culture, or cell lysate from which cells or cell debris has been substantially removed by, for example, filtration or centrifugation.

As used herein “bioreactor” takes its art recognized meaning and refers to a chamber designed for the controlled growth of a cell culture. The bioreactor can be of any size as long as it is useful for the culturing of cells, e.g., mammalian cells. Typically, the bioreactor will be at least 30 ml and may be at least 1, 10, 100, 250, 500, 1000, 2500, 5000, 8000, 10,000, 12,0000 liters or more, or any intermediate volume. The internal conditions of the bioreactor, including but not limited to pH and temperature, are typically controlled during the culturing period. A suitable bioreactor may be composed of (i.e., constructed of) any material that is suitable for holding cell cultures suspended in media under the culture conditions and is conductive to cell growth and viability, including glass, plastic or metal; the material(s) should not interfere with expression or stability of a protein of interest. One of ordinary skill in the art will be aware of, and will be able to choose, suitable bioreactors for use in practicing the present invention.

As used herein, a “mixture” comprises a protein of interest (for which purification is desired) and one or more contaminant, i.e., impurities. In one embodiment, the mixture is produced from a host cell or organism that expresses the protein of interest (either naturally or recombinantly). Such mixtures include, for example, cell cultures, cell lysates, and clarified bulk (e.g., clarified cell culture supernatant).

As used herein, the terms “separating” and “purifying” are used interchangeably, and refer to the selective removal of contaminants from a mixture containing a protein of interest (e.g., an antibody).

As used herein the term “contaminant” is used in its broadest sense to cover any undesired component or compound within a mixture. In cell cultures, cell lysates, or clarified bulk (e.g., clarified cell culture supernatant), contaminants include, for example, host cell nucleic acids (e.g., DNA) and host cell proteins present in a cell culture medium. Host cell contaminant proteins include, without limitation, those naturally or recombinantly produced by the host cell, as well as proteins related to or derived from the protein of interest (e.g., proteolytic fragments) and other process related contaminants. In certain embodiments, the contaminant precipitate is separated from the cell culture using an art-recognized means, such as centrifugation, sterile filtration, depth filtration and tangential flow filtration.

As used herein “centrifugation” is a process that involves the use of the centrifugal force for the sedimentation of heterogeneous mixtures with a centrifuge, used in industry and in laboratory settings. This process is used to separate two immiscible liquids. For example, in a method of the present invention, centrifugation can be used to remove a contaminant precipitation from a mixture, including without limitation, a cell culture or clarified cell culture supernatant or capture-column captured elution pool.

As used herein “sterile filtration” is a filtration method that use membrane filters, which are typically a filter with pore size 0.2 μm to effectively remove microorganisms or small particles. For example, in a method of the present invention, sterile filtration can be used to remove a contaminant precipitate from a mixture, including without limitation, a cell culture or clarified cell culture supernatant or capture-column captured elution pool.

As used herein “depth filtration” is a filtration method that uses depth filters, which are typically characterized by their design to retain particles due to a range of pore sizes within a filter matrix. The depth filter's capacity is typically defined by the depth, e.g., 10 inch or 20 inch of the matrix and thus the holding capacity for solids. For example, in a method of the present invention, depth filtration can be used to remove a contaminant precipitate from a mixture, including without limitation, a cell culture or clarified cell culture supernatant or capture-column captured elution pool.

As used herein, the term “tangential flow filtration” refers to a filtration process in which the sample mixture circulates across the top of a membrane, while applied pressure causes certain solutes and small molecules to pass through the membrane. For example, in a method of the present invention, tangential flow filtration can be used to remove a contaminant precipitate from a mixture, including without limitation, a cell culture or clarified cell culture supernatant or capture-column captured elution pool.

As used herein the term “chromatography” refers to the process by which a solute of interest, e.g., a protein of interest, in a mixture is separated from other solutes in the mixture by percolation of the mixture through an adsorbent, which adsorbs or retains a solute more or less strongly due to properties of the solute, such as pI, hydrophobicity, size and structure, under particular buffering conditions of the process. In a method of the present invention, chromatography can be used to remove contaminants after the precipitate is removed from a mixture, including without limitation, a cell culture or clarified cell culture supernatant or capture-column captured elution pool.

The terms “ion-exchange” and “ion-exchange chromatography” refer to a chromatographic process in which an ionizable solute of interest (e.g., a protein of interest in a mixture) interacts with an oppositely charged ligand linked (e.g., by covalent attachment) to a solid phase ion exchange material under appropriate conditions of pH and conductivity, such that the solute of interest interacts non-specifically with the charged compound more or less than the solute impurities or contaminants in the mixture. The contaminating solutes in the mixture can be washed from a column of the ion exchange material or are bound to or excluded from the resin, faster or slower than the solute of interest. “Ion-exchange chromatography” specifically includes cation exchange, anion exchange, and mixed mode chromatographies.

The phrase “ion exchange material” refers to a solid phase that is negatively charged (i.e., a cation exchange resin or membrane) or positively charged (i.e., an anion exchange resin or membrane). In one embodiment, the charge can be provided by attaching one or more charged ligands (or adsorbents) to the solid phase, e.g., by covalent linking. Alternatively, or in addition, the charge can be an inherent property of the solid phase (e.g., as is the case for silica, which has an overall negative charge).

A “cation exchange resin” refers to a solid phase which is negatively charged, and which has free cations for exchange with cations in an aqueous solution passed over or through the solid phase. Any negatively charged ligand attached to the solid phase suitable to form the cation exchange resin can be used, e.g., a carboxylate, sulfonate and others as described below. Commercially available cation exchange resins include, but are not limited to, for example, those having a sulfonate based group (e.g., MonoS, MiniS, Source 15S and 30S, SP Sepharose Fast Flow™, SP Sepharose High Performance from GE Healthcare, Toyopearl SP-650S and SP-650M from Tosoh, Macro-Prep High S from BioRad, Ceramic HyperD S, Trisacryl M and LS SP and Spherodex LS SP from Pall Technologies); a sulfoethyl based group (e.g., Fractogel SE, from EMD, Poros S-10 and S-20 from Applied Biosystems); a sulphopropyl based group (e.g., TSK Gel SP 5PW and SP-5PW-HR from Tosoh, Poros HS-20 and HS 50 from Applied Biosystems); a sulfoisobutyl based group (e.g., Fractogel EMD SO₃⁻ from EMD); a sulfoxyethyl based group (e.g., SE52, SE53 and Express-Ion S from Whatman), a carboxymethyl based group (e.g., CM Sepharose Fast Flow from GE Healthcare, Hydrocell CM from Biochrom Labs Inc., Macro-Prep CM from BioRad, Ceramic HyperD CM, Trisacryl M CM, Trisacryl LS CM, from Pall Technologies, Matrx Cellufine C500 and C200 from Millipore, CM52, CM32, CM23 and Express—Ion C from Whatman, Toyopearl CM-650S, CM-650M and CM-650C from Tosoh); sulfonic and carboxylic acid based groups (e.g., BAKERBOND Carboxy-Sulfon from J.T. Baker); a carboxylic acid based group (e.g., WP CBX from J.T Baker, DOWEX MAC-3 from Dow Liquid Separations, Amberlite Weak Cation Exchangers, DOWEX Weak Cation Exchanger, and Diaion Weak Cation Exchangers from Sigma-Aldrich and Fractogel EMD COO-from EMD); a sulfonic acid based group (e.g., Hydrocell SP from Biochrom Labs Inc., DOWEX Fine Mesh Strong Acid Cation Resin from Dow Liquid Separations, UNOsphere S, WP Sulfonic from J. T. Baker, Sartobind S membrane from Sartorius, Amberlite Strong Cation Exchangers, DOWEX Strong Cation and Diaion Strong Cation Exchanger from Sigma-Aldrich); and a orthophosphate based group (e.g., P11 from Whatman).

An “anion exchange resin” refers to a solid phase which is positively charged, thus having one or more positively charged ligands attached thereto. Any positively charged ligand attached to the solid phase suitable to form the anionic exchange resin can be used, such as quaternary amino groups Commercially available anion exchange resins include DEAE cellulose, Poros PI 20, PI 50, HQ 10, HQ 20, HQ 50, D 50 from Applied Biosystems, Sartobind Q from Sartorius, MonoQ, MiniQ, Source 15Q and 30Q, Q, DEAE and ANX Sepharose Fast Flow, Q Sepharose high Performance, QAE SEPHADEX™ and FAST Q SEPHAROSE™ (GE Healthcare), WP PEI, WP DEAM, WP QUAT from J.T. Baker, Hydrocell DEAE and Hydrocell QA from Biochrom Labs Inc., UNOsphere Q, Macro-Prep DEAE and Macro-Prep High Q from Biorad, Ceramic HyperD Q, ceramic HyperD DEAE, Trisacryl M and LS DEAE, Spherodex LS DEAE, QMA Spherosil LS, QMA Spherosil M and Mustang Q from Pall Technologies, DOWEX Fine Mesh Strong Base Type I and Type II Anion Resins and DOWEX MONOSPHER E 77, weak base anion from Dow Liquid Separations, Intercept Q membrane, Matrex Cellufine A200, A500, Q500, and Q800, from Millipore, Fractogel EMD TMAE, Fractogel EMD DEAE and Fractogel EMD DMAE from EMD, Amberlite weak strong anion exchangers type I and II, DOWEX weak and strong anion exchangers type I and II, Diaion weak and strong anion exchangers type I and II, Duolite from Sigma-Aldrich, TSK gel Q and DEAE 5PW and 5PW-HR, Toyopearl SuperQ-650S, 650M and 650C, QAE-550C and 650S, DEAE-650M and 650C from Tosoh, QA52, DE23, DE32, DE51, DE52, DE53, Express-Ion D and Express-Ion Q from Whatman, and Sartobind Q (Sartorius corporation, New York, USA).

A “mixed mode ion exchange resin” or “mixed mode” refers to a solid phase which is covalently modified with cationic, anionic, and/or hydrophobic moieties. Examples of mixed mode ion exchange resins include BAKERBOND ABX™ (J. T. Baker; Phillipsburg, N.J.), ceramic hydroxyapatite type I and II and fluoride hydroxyapatite (BioRad; Hercules, Calif.) and MEP and MBI HyperCel (Pall Corporation; East Hills, N.Y.).

A “hydrophobic interaction chromatography resin” refers to a solid phase which is covalently modified with phenyl, octyl, or butyl chemicals. Hydrophobic interaction chromatography is a separation technique that uses the properties of hydrophobicity to separate proteins from one another. In this type of chromatography, hydrophobic groups such as, phenyl, octyl, or butyl are attached to the stationary column. Proteins that pass through the column that have hydrophobic amino acid side chains on their surfaces are able to interact with and bind to the hydrophobic groups on the column. Examples of hydrophobic interaction chromatography resins include: (1) Butyl FF, Butyl HP, Octyl FF, Phenyl FF, Phenyl HP, Phenyl FF (high sub), Phenyl FF (low sub), Capto Phenyl ImpRes, Capto Phenyl (high sub), Capto Octyl, Capto ButyllmpRes, Capto Butyl (GE Healthcare, Uppsala, Sweden); (2) Toyopearl Super Butyl-550C, Toyopearl Hexyl-650C, Butyl-650C, Phenyl-650C, Butyl 600 M, Phenyl-600M, PPG-600M, Butyl-650M, Phenyl-650M, Ether-650M, Butyl-650S, Phenyl-650S, Ether-650S, TSKgel Pheny-5PW, TSKgel Ether-5PW (Tosoh Bioscience, Tokyo, Japan); (3) Macro-Prep-butyl, Macro-Prep-methyl (Bio-Rad); and (4) Sartobind Phenyl (Sartorius corporation, New York, USA).

### II. Proteins of Interest

In certain aspects, methods of the present invention may be used to purify any protein of interest including, but not limited to, proteins having pharmaceutical, diagnostic, agricultural, and/or any of a variety of other properties that are useful in commercial, experimental or other applications. In addition, a protein of interest can be a protein therapeutic. In certain embodiments, proteins purified using methods of the present invention may be processed or modified. For example, a protein of interest in accordance with the present invention may be glycosylated.

Thus, the present invention may be used to culture cells for production of any therapeutic protein, such as pharmaceutically or commercially relevant enzymes, receptors, receptor fusion proteins, antibodies (e.g., monoclonal or polyclonal antibodies), antigen-binding fragments of an antibody, Fc fusion proteins, cytokines, hormones, regulatory factors, growth factors, coagulation/clotting factors, or antigen-binding agents. The above list of proteins is merely exemplary in nature, and is not intended to be a limiting recitation. One of ordinary skill in the art will know that other proteins can be produced in accordance with the present invention, and will be able to use methods disclosed herein to produce such proteins.

In one particular embodiment of the invention, the protein purified using the method of the invention is an antibody. The term “antibody” is used in the broadest sense to cover monoclonal antibodies (including full length monoclonal antibodies), polyclonal antibodies, multispecific antibodies (e.g., bispecific antibodies), antibody fragments, immunoadhesins and antibody-immunoadhesin chimerias.

An “antibody fragment” includes at least a portion of a full length antibody and typically an antigen binding or variable region thereof. Examples of antibody fragments include Fab, Fab′, F(ab′)₂, and Fv fragments; single-chain antibody molecules; diabodies; linear antibodies; and multispecific antibodies formed from engineered antibody fragments.

The term “monoclonal antibody” is used in the conventional sense to refer to an antibody obtained from a population of substantially homogeneous antibodies such that the individual antibodies comprising the population are identical except for possible naturally occurring mutations that may be present in minor amounts. Monoclonal antibodies are highly specific, being directed against a single antigenic site. This is in contrast with polyclonal antibody preparations which typically include varied antibodies directed against different determinants (epitopes) of an antigen, whereas monoclonal antibodies are directed against a single determinant on the antigen. The term “monoclonal”, in describing antibodies, indicates the character of the antibody as being obtained from a substantially homogeneous population of antibodies, and is not to be construed as requiring production of the antibody by any particular method. For example, monoclonal antibodies used in the present invention can be produced using conventional hybridoma technology first described by Kohler et al., Nature 256:495 (1975), or they can be made using recombinant DNA methods (see, e.g., U.S. Pat. No. 4,816,567). Monoclonal antibodies can also be isolated from phage antibody libraries, e.g., using the techniques described in Clackson et al., Nature 352:624-628 (1991); Marks et al., J. Mol. Biol. 222:581-597 (1991); and U.S. Pat. Nos. 5,223,409; 5,403,484; 5,571,698; 5,427,908 5,580,717; 5,969,108; 6,172,197; 5,885,793; 6,521,404; 6,544,731; 6,555,313; 6,582,915; and 6,593,081).

The monoclonal antibodies described herein include “chimeric” and “humanized” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)). “Humanized” forms of non-human (e.g., murine) antibodies are chimeric antibodies which contain minimal sequence derived from non-human immunoglobulin. For the most part, humanized antibodies are human immunoglobulins (recipient antibody) in which the hypervariable region residues of the recipient are replaced by hypervariable region residues from a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

Chimeric or humanized antibodies can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art (see e.g., U.S. Pat. No. 5,225,539 to Winter, and U.S. Pat. Nos. 5,530,101; 5,585,089; 5,693,762 and 6,180,370 to Queen et al.).

The monoclonal antibodies described herein also include “human” antibodies, which can be isolated from various sources, including, e.g., from the blood of a human patient or recombinantly prepared using transgenic animals. Examples of such transgenic animals include KM-Mouse® (Medarex, Inc., Princeton, N.J.) which has a human heavy chain transgene and a human light chain transchromosome (see WO 02/43478), Xenomouse® (Abgenix, Inc., Fremont Calif.; described in, e.g., U.S. Pat. Nos. 5,939,598; 6,075,181; 6,114,598; 6,150,584 and 6,162,963 to Kucherlapati et al.), and HuMAb-Mouse® (Medarex, Inc.; described in, e.g., Taylor, L. et al. (1992) Nucleic Acids Research 20:6287-6295; Chen, J. et al. (1993) International Immunology 5: 647-656; Tuaillon et al. (1993) Proc. Natl. Acad. Sci. USA 90:3720-3724; Choi et al. (1993) Nature Genetics 4:117-123; Chen, J. et al. (1993) EMBO J. 12: 821-830; Tuaillon et al. (1994) J. Immunol. 152:2912-2920; Taylor, L. et al. (1994) International Immunology 6: 579-591; and Fishwild, D. et al. (1996) Nature Biotechnology 14: 845-851, U.S. Pat. Nos. 5,545,806; 5,569,825; 5,625,126; 5,633,425; 5,789,650; 5,877,397; 5,661,016; 5,814,318; 5,874,299; and 5,770,429; 5,545,807; and PCT Publication Nos. WO 92/03918, WO 93/12227, WO 94/25585, WO 97/13852, WO 98/24884 and WO 99/45962, WO 01/14424 to Korman et al.). Human monoclonal antibodies of the invention can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

In certain specific embodiments, the present invention provides methods of purifying an anti-IP10 monoclonal antibody. Preferably, such methods are used to purify monomeric antibodies from aggregate forms of the antibody (e.g., dimers, multimers, intermediate aggregate species).

In certain aspects, the anti-IP10 monoclonal antibody comprises heavy chain CDR1, CDR2, and CDR3 amino acid sequences of SEQ ID NOs: 1, 2, and 3, respectively. In certain aspects, the anti-IP10 monoclonal antibody comprises light chain CDR1, CDR2, and CDR3 amino acid sequences of SEQ ID NOs: 6, 7, and 8, respectively. To illustrate, the anti-IP10 monoclonal antibody comprises heavy chain CDR1, CDR2, and CDR3 sequences of SEQ ID NOs: 1, 2, and 3, and light chain CDR1, CDR2, and CDR3 sequences of SEQ ID NOs: 6, 7, and 8, respectively. To illustrate, the anti-IP10 monoclonal antibody comprises a heavy variable region sequence and a light chain variable region sequence of SEQ ID NOs: 4 and 9, respectively. To illustrate, the anti-IP10 monoclonal antibody comprises the full-length heavy chain amino acid sequence and the full-length light chain amino acid sequence of SEQ ID NOs: 5 and 10, respectively.

The table below lists the amino acid sequences of an exemplary anti-IP10 mAb.

| | | |
|---|---|---|
| 1 | VH CDR1 a.a. | EYGMH |
| 2 | VH CDR2 a.a. | VIGFAGLIKGYADSVKG |
| 3 | VH CDR3 a.a. | EGAGSNIYYYYGMDV |
| 4 | VH a.a. | QVQLVESGGGVVQPGRSLRLSCAAS |
| | | GFTFSEYGMHWVRQAPGKGLEWVAV |
| | | IGFAGLIKGYADSVKGRFTISRDNS |
| | | KNTLYLQMNSLRAEDTAVYYCAREG |
| | | AGSNIYYYYGMDVWGQGTTVTVSS |
| 5 | Full-length | QVQLVESGGGVVQPGRSLRLSCAAS |
| | heavy chain | GFTFSEYGMHWVRQAPGKGLEWVAV |
| | a.a. | IGFAGLIKGYADSVKGRFTISRDNS |
| | | KNTLYLQMNSLRAEDTAVYYCAREG |
| | | AGSNIYYYYGMDVWGQGTTVTVSSA |
| | | STKGPSVFPLAPSSKSTSGGTAALG |
| | | CLVKDYFPEPVTVSWNSGALTSGVH |
| | | TFPAVLQSSGLYSLSSVVTVPSSSL |
| | | GTQTYICNVNHKPSNTKVDKRVEPK |
| | | SCDKTHTCPPCPAPELLGGPSVFLF |
| | | PPKPKDTLMISRTPEVTCVVVDVSH |
| | | EDPEVKFNWYVDGVEVHNAKTKPRE |
| | | EQYNSTYRVVSVLTVLHQDWLNGKE |
| | | YKCKVSNKALPAPIEKTISKAKGQP |
| | | REPQVYTLPPSREEMTKNQVSLTCL |
| | | VKGFYPSDIAVEWESNGQPENNYKT |
| | | TPPVLDSDGSFFLYSKLTVDKSRWQ |
| | | QGNVFSCSVMHEALHNHYTQKSLSL |
| | | SPGK |
| 6 | VL CDR1 a.a. | RASQSVSSSYL |
| 7 | VL CDR2 a.a. | GASSRAT |
| 8 | VL CDR3 a.a. | QQYGSSPIFT |
| 9 | VL a.a. | EIVLTQSPGTLSLSPGERATLSCRA |
| | | SQSVSSSYLAWYQQKPGQAPRLLIY |
| | | GASSRATGIPDRFSGSGSGTDFTLT |
| | | ISRLEPEDFAVYYCQQYGSSPIFTF |
| | | GPGTKVDIK |
| 10 | Full-length | EIVLTQSPGTLSLSPGERATLSCRA |
| | light chain | SQSVSSSYLAWYQQKPGQAPRLLIY |
| | a.a. | GASSRATGIPDRFSGSGSGTDFTLT |
| | | ISRLEPEDFAVYYCQQYGSSPIFTF |
| | | GPGTKVDIKRTVAAPSVFIFPPSDE |
| | | QLKSGTASVVCLLNNFYPREAKVQW |
| | | KVDNALQSGNSQESVTEQDSKDSTY |
| | | SLSSTLTLSKADYEKHKVYACEVTH |
| | | QGLSSPVTKSFNRGEC |

### III. Mixtures Containing a Protein of Interest

The methods of the invention can be applied to any mixture containing a protein of interest. In one embodiment, the mixture is obtained from or produced by living cells that express the protein to be purified (e.g., naturally or by genetic engineering). Optionally, the cells in a cell culture include cells transfected with an expression construct containing a nucleic acid that encodes a protein of interest. Methods of genetically engineering cells to produce proteins are well known in the art. See e.g., Ausabel et al., eds. (1990), Current Protocols in Molecular Biology (Wiley, New York) and U.S. Pat. Nos. 5,534,615 and 4,816,567, each of which are specifically incorporated herein by reference. Such methods include introducing nucleic acids that encode and allow expression of the protein into living host cells. These host cells can be bacterial cells, fungal cells, insect cells or, preferably, animal cells grown in culture. Bacterial host cells include, but are not limited to E. coli cells. Examples of suitable E. coli strains include: HB101, DH5α, GM2929, JM109, KW251, NM538, NM539, and any E. coli strain that fails to cleave foreign DNA. Fungal host cells that can be used include, but are not limited to, Saccharomyces cerevisiae, Pichia pastoris and Aspergillus cells. Insect cells that can be used include, but are not limited to, Bombyx mori, Mamestra drassicae, Spodoptera frupperda, Trichoplusia ni, Drosophilia melanogaster.

A number of mammalian cell lines are suitable host cells for expression of proteins of interest. Mammalian host cell lines include, for example, COS, PER.C6, TM4, VERO076, DXB11, MDCK, BRL-3A, W138, Hep G2, MMT, MRC 5, FS4, CHO, 293T, A431, 3T3, CV-1, C3H10T1/2, Colo205, 293, HeLa, L cells, BHK, HL-60, FRhL-2, U937, HaK, Jurkat cells, Rat2, BaF3, 32D, FDCP-1, PC12, M1x, murine myelomas (e.g., SP2/0 and NS0) and C2C12 cells, as well as transformed primate cell lines, hybridomas, normal diploid cells, and cell strains derived from in vitro culture of primary tissue and primary explants. New animal cell lines can be established using methods well known by those skilled in the art (e.g., by transformation, viral infection, and/or selection). Any eukaryotic cell that is capable of expressing the protein of interest may be used in the disclosed cell culture methods. Numerous cell lines are available from commercial sources such as the American Type Culture Collection (ATCC). In one embodiment of the invention, the cell culture, e.g., the large-scale cell culture, employs hybridoma cells. The construction of antibody-producing hybridoma cells is well known in the art. In one embodiment of the invention, the cell culture, e.g., the large-scale cell culture, employs CHO cells to produce the protein of interest such as an antibody (see, e.g., WO 94/11026). Various types of CHO cells are known in the art, e.g., CHO-K1, CHO-DG44, CHO-DXB11, CHO/dhff and CHO-S.

In certain embodiments, the present invention contemplates, prior to purifying a protein of interest from a cell culture, monitoring particular conditions of the growing cell culture. Monitoring cell culture conditions allows for determining whether the cell culture is producing the protein of interest at adequate levels. For example, small aliquots of the culture are periodically removed for analysis in order to monitor certain cell culture conditions. Cell culture conditions to be monitored include, but not limited to, temperature, pH, cell density, cell viability, integrated viable cell density, lactate levels, ammonium levels, osmolality, and titer of the expressed protein. Numerous techniques are well known to those of skill in the art for measuring such conditions/criteria. For example, cell density may be measured using a hemocytometer, an automated cell-counting device (e.g., a Coulter counter, Beckman Coulter Inc., Fullerton, Calif.), or cell-density examination (e.g., CEDEX®, Innovatis, Malvern, Pa.). Viable cell density may be determined by staining a culture sample with Trypan blue. Lactate and ammonium levels may be measured, e.g., with the BioProfile 400 Chemistry Analyzer (Nova Biomedical, Waltham, Mass.), which takes real-time, online measurements of key nutrients, metabolites, and gases in cell culture media. Osmolality of the cell culture may be measured by, e.g., a freezing point osmometer. HPLC can be used to determine, e.g., the levels of lactate, ammonium, or the expressed protein. In one embodiment of the invention, the levels of expressed protein can be determined by using, e.g., protein A HPLC. Alternatively, the level of the expressed protein can be determined by standard techniques such as Coomassie staining of SDS-PAGE gels, Western blotting, Bradford assays, Lowry assays, biuret assays, and UV absorbance. Optionally, the present invention may include monitoring the post-translational modifications of the expressed protein, including phosphorylation and glycosylation.

In a specific embodiment, methods of the present invention comprise effectively removing contaminants from a mixture (e.g., a cell culture, cell lysate or clarified bulk) which contains a high concentration of a protein of interest (e.g., an antibody). For example, the concentration of a protein of interest may range from about 0.5 to about 50 mg/ml (e.g., 0.5, 1, 5, 10, 15, 20, 25, 30, 35, 40, 45 or 50 mg/ml).

Preparation of mixtures initially depends on the manner of expression of the protein. Some cell systems directly secrete the protein (e.g., an antibody) from the cell into the surrounding growth media, while other systems retain the antibody intracellularly. For proteins produced intracellularly, the cell can be disrupted using any of a variety of methods, such as mechanical shear, osmotic shock, and enzymatic treatment. The disruption releases the entire contents of the cell into the homogenate, and in addition produces subcellular fragments which can be removed by centrifugation or by filtration. A similar problem arises, although to a lesser extent, with directly secreted proteins due to the natural death of cells and release of intracellular host cell proteins during the course of the protein production run.

In one embodiment, cells or cellular debris are removed from the mixture, for example, to prepare clarified bulk. The methods of the invention can employ any suitable methodology to remove cells or cellular debris. If the protein is produced intracellularly, as a first step, the particulate debris, either host cells or lysed fragments, can be removed, for example, by a centrifugation or filtration step in order to prepare a mixture which is then subjected to purification according the methods described herein (i.e., from which a protein of interest is purified). If the protein is secreted into the medium, the recombinant host cells may be separated from the cell culture medium by, e.g., centrifugation, tangential flow filtration or depth filtration, in order to prepare a mixture from which a protein of interest is purified.

In another embodiment, cell culture or cell lysate is used directly without first removing the host cells. Indeed, the methods of the invention are particularly well suited to using mixtures comprising a secreted protein and a suspension of host cells.

The present disclosure is further illustrated by the following examples, which should not be construed as further limiting. The contents of all figures and all references, patents and published patent applications cited throughout this application are expressly incorporated herein by reference in their entireties.

### Example 1

### Characterization and Removal of a Monoclonal Antibody Aggregate Containing Four-Light Chains

### Introduction

Protein aggregation is an important quality attribute due to its effect on potency and pharmacokinetics [1-4]. Despite extensive efforts to minimize negative effects on the molecules and implement effective control strategy during protein development, the formation of undesired high molecular weight species and aggregates cannot be avoided completely [5, 6]. Therefore, aggregation level needs to be closely monitored through entire upstream cell culture and downstream purification process.

A platform approach that includes Protein A (ProA) as the capture step and ion (anion or cation) exchange chromatogram (IEX) as the polishing step has been widely utilized in mAb purification [7-9]. The initial ProA was to remove the bulk of impurities present in the clarified harvest. The IEX was to remove product impurities such as aggregates and process impurities including host cell proteins (HCP) and residual host cell DNA. In most cases, a cation exchange chromatography (CEX) bind and elute mode using a salt step gradient can be employed to remove product aggregates due to their increase of the surface charge compared to monomer [10-12]. Besides IEX, hydrophobic interaction chromatography and mixed-mode chromatography are also commonly utilized in polishing chromatographic step to remove aggregates [20, 29-33]. However, not all aggregate species behave the same. In general, larger aggregates may show relatively higher hydrophobicity and more surface charge, therefore more readily to be removed by IEX or HIC. In recent years increasing number of studies on an intermediate species, an aggregate species between the dimeric and monomeric species has been reported [13-15]. Gomez [13, 14] discovered a three-light chain (3LC) species and investigated the upstream factors that influenced the formation of the 3LC species. Furthermore, the intermediate species was found to be more challenging to remove using the conventional platform approach. Wollacott [15] characterized the intermediate species and developed a hydrophobic interaction chromatography (HIC) process to efficiently remove the species. However, 12% ethanol was needed in the elution buffer in order to overcome the drawback of the large elution volume. Chen [17] evaluated different mixed-mode resins and designed a new platform using protein A-MEP-CHT to effectively remove high levels of aggregates. Gao [18] found that the combination of hydrophobic interaction and electrostatic interaction is important for the effective aggregate removal with mixed-mode resins. However, the process and manufacturing understanding of these resins was limited.

Here, Applicants report an intermediate species using SE-HPLC. Further characterization revealed the 200 kDa intermediate species contains four-light chains, two from a full mAb and two from a LC dimer. Contrary to most aggregates, the 4LC intermediate species was found to be less hydrophobic and less electrostatic than the monomer species, which poses a big challenge in aggregate removal in using CEX and HIC bind/elute mode. In our initial process development, the intermediate species was not removed by the CEX bind and elution mode. Several attempts have been made to use various types of resins without much success. However, in recent years, high-throughput screening (HTS) of chromatographic conditions using batch-binding in 96-well filterplates greatly enhances the efficiency of protein purification development. Kelley [19] used high-throughput screening (HTS) system to accelerate process development by evaluating the protein partition coefficients to estimate the characteristics charge of the resin-protein interaction. Therefore, Applicants applied an HTS system and measured the adsorption isotherm for total 56 different pH and salt combinations on the Poros XS resin. By calculating the partition coefficients for different species, an optimal condition to effectively remove the intermediate species was determined. Applicants then applied the optimal condition to a Poros XS CEX column and further developed a polishing process.

Moreover, it was found that the isoelectric point (pI) of the intermediate species was about 0.4 pH unit lower than the monomer. Since pH gradient had been widely used in analytical scale in separation of charge variants [21-28], the same principle using a pH gradient can be applied to separate the intermediate species from monomer based on their different pI values. Therefore, in this work, we modulated the buffer pH to alter the surface charge of the protein, and thereby influenced selectivity between these species and monomer. Under the bind elution mode, the running condition was optimized to remove the intermediate species using a high pH wash buffer and to clear other aggregate species using a buffer with high salt. Since the intermediate species was the main focus of the study, a DOE experiment was designed to further confirm the wash buffer conditions including wash buffer pH, wash buffer salt concentration, and wash buffer volume. The load amount was also incorporated into the study since it has shown impact on aggregate clearance in other occasions. As a result, Applicants developed a robust and effective purification to remove the challenging intermediate species with monomer purity greater than 99% and step yield over 80%.

### Materials and Methods

### Materials

1. The anti-IP10 monoclonal antibody was expressed by Chinese Hamster Ovary (CHO) cell lines. The cell culture materials were harvested by using two stage Zeta Plus™ depth filters (10SP05A/90ZB05A, 3M, USA) followed by 0.2 μM sterile filter capsule (Sartorius, USA).

2. Resins

Capture step Protein A resin is Mabselect Protein A affinity resin from GE Healthcare (Piscataway, N.J., USA). The CEX resin is Poros XS from Life Technologies (Carlsbad, Calif., USA). Other resins used in this study were Capto Phenyl, Tosoh Butyl, Phenyl Sepherose, Capto MMC, Capto Adhere ImPres, MEP HyperCel, FractoGel MED SO₃⁻ (Merck KGaA, Darmstadt, Germany).

3. Chromatography Purification Process

All chemical reagents were from Sigma (St. Louis, Mo., USA) and J.T. Baker (Mallinkrodt Baker, Phillipsburg, N.J., USA) unless otherwise noted. Chromatography separations were performed on AKTA Avant system from GE Healthcare (Piscataway, N.J., USA) controlled by Unicorn 7.0 software.

All chromatography studies used constant residence time of 4 minutes. Columns were packed to bed heights of 10-20 cm following manufacturer's recommendations and evaluated based on HETP and peak asymmetry factor. The detailed operation conditions were illustrated in the results section or in figure legend. The product purity was evaluated by analytical SEC-HPLC.

### Preparative SEC

To separate the intermediate high molecular weight species, the Protein A elution from the anti-IP10 mAb was injected onto a preparative SEC column (21.5 mm×30 cm) from Tosoh Bioscience (King of Prussia, Pa., USA). The injection volume was 0.5 mL with a total loading of 7-8 mg of protein per run. The running buffer is 0.1 M potassium phosphate and 0.15 M sodium chloride, pH 6.8. Fractions were collected and pools were made according to the elution profile.

### High-Throughput Screening System

A Tecan Genesis 150 (Tecan US, Research Triangle Park, N.C.) was used for liquid and resin handling. A 96-well filterplate (Innovative Microplate, Billerica, Mass., p/n F20022), with a 0.45 mm PVDF membrane was used to incubate the resin, protein, and solution mixtures. The filterplate was centrifuged at 1200 g to separate the supernatant solution from the resin. The filtrate was captured in the collection plate which was stacked beneath the filterplate. The samples the collection plate were then analyzed by a UV-vis spectrophotometer in a 96-well format. The samples were also analyzed SE-HPLC for aggregation. All experiments were performed at room temperature.

### Analytical Assays

1. Size-Exclusion HPLC (SEC)

Size Exclusion HPLC (SEC) was used for the quantitative analysis of monomer, High Molecular Weight (HMW), and Low Molecular Weight (LMW) species of each size variant fractions. Samples were analyzed using a Tosoh Bioscience G3000 SW_{XL} column (Part #: 08541, King of Prussia, Pa.). with a flow rate of 1.0 mL/minute using 0.1 M potassium phosphate and 0.15M sodium chloride, pH 6.8 as the mobile phase. The peaks were detected by UV absorption at 280 nm. The results were reported as the area percentage for the monomer, HMW, and LMW species.

2. Chip-Based CE (Caliper)

The mAb HMW species were analyzed using the Caliper LabChip® GXII instrument (Perkin Elmer, Waltham, Mass.) in both non-reduced and reduced conditions. The regular microchip-based electrophoresis has been described in details elsewhere with minor modifications. Briefly, 2 μL of antibody at 2 mg/mL was mixed with 14 μL of sample buffer. The sample buffer was prepared by mixing 700 μL of PerkinElmer HT Protein Express sample buffer with either 24.5 μL of BME (for reducing assay) or 35 μL of 0.5 M iodoacetamide (IAM, for the non-reducing assay). The samples were incubated at 90° C. for 5 min. After cooling to room temperature, 70 μL of water was added to each sample before loading onto the instrument. The chip was prepared according to the manufacturer's instruction. The samples were analyzed using the built-in script provided by PerkinElmer.

3. Hydrophobic Interaction HPLC (HIC)

A HPLC-based HIC method (TSKgel Butyl-NPR, 4.6 mm×10 cm, Tosh Bioscience) was used to determine the relative hydrophobicity of each aggregate species. A linear gradient method with a flow rate of 0.5 mL/min was used with mobile phase A (0.1 M potassium phosphate, 0.15 M sodium chloride, pH 6.8, with 2 M ammonium sulfate) and mobile phase B (0.1 M potassium phosphate, 0.15 M sodium chloride, pH 6.8). The fractionated aggregate species were injected into the HIC column with total loading about 30 pg.

4. Cationic Exchange HPLC

A weak cationic exchange column (WCX-10, 2.5 mm×30 cm, Dionex) was used in determine the overall relative net charge of each fractionated aggregate. A linear gradient was used with mobile phase A (20 mM acetate, pH 5.5) and mobile phase B (20 mM acetate, 1.0 M sodium chloride, pH 5.5) at a flow rate of 0.25 mL/min. The peaks were detected using a UV detector at 280 nm.

5. Imaged Capillary Electrophoresis (iCE)

All mAbs samples were diluted to 2.5 g/L. Diluted protein samples (20 μL) was mixed with prepared Ampholyte solution (180 μL) containing 1.0% methyl cellulose (MC) solution (70 μL), Pharmalyte 3-10 (8 μL), 8M urea (50 μL), pI markers 4.22 and 9.46 (1 μL each), and water (50 μL). The sample was mixed well and was injected to the iCIEF instrument.

The sample is pre-focused at 1500V and then focused at 3000V. The IEF process within the separation capillary was recorded using CCD camera to acquire UV light absorption image every 30 seconds. The pI values of the peaks are calculated using a two-point calibration with the pI markers using iCE CFR software 4.1 (ProteinSimple, San Jose, Calif. USA). The quantitative analysis of peak percentage of each peak was done in Empower 3 (Waters, Milford, Mass.).

6. SEC-MALS

The antibody species and their complex with Protein A were analyzed by SEC using a tandem column of TSKgel G 3000SWxl (TOSOH Bioscience) on Waters HPLC 2695 Alliance. The mobile phase is 100 mM potassium phosphate, 150 mM NaCl, pH 6.8 buffer, applied at a flow rate of 0.5 ml/min. The signals of UV, light scattering and refractive index were respectively monitored by 2489 UV/Vis detector (Wyatt), miniDAWN TREOS (Wyatt) and Optilab T-rEX (Wyatt). The data was processed by ASTRA 6.1 (Wyatt).

6. Native Nano ESI/MS

7. ELISA for HCP

Host cell protein was detected using a commercial microtiter plate ELISA method specific for the hybridoma cell line NS/0 (Cygnus Technologies, NC, USA). Samples were diluted with sample dilution buffer (consisting of 2 mg/ml IgG in phosphate buffered saline (PBS), pH 7.0) employed with the kit and analyzed according to the manufacturer's standard assay protocol. A plate spectrophotometer (Tecan Safire II, Ser. No. 501000005, Tecan AG, M\ddot{ } annedorf, Switzerland) was set to dual wave length at 450 nm/630 nm (test/reference) to read the colorimetric reaction of standards and samples.

### Results

### 1. Initial CEX Run

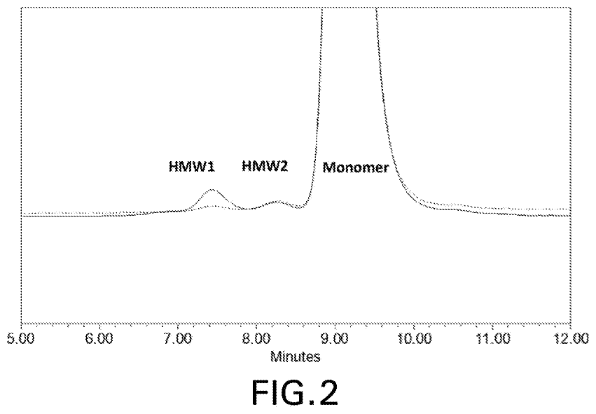

The initial development work was carried out using a platform process including a Protein A chromatography as the capture step and a cation exchange chromatography (CEX) as the polishing step. The mAb protein A elution pool was viral inactivated under low pHs with a range of 3.5-3.7 followed by neutralization to pH 5.5. A typical SEC chromatogram of the neutralized protein A elution pool was presented in FIG. 2 (solid line) with overall aggregation species at 5%. The two aggregation species were named HMW1 and HMW2. The protein A pool was loaded onto a CEX column (1 cm×20 cm) at 25 g protein/L (resin) loading.

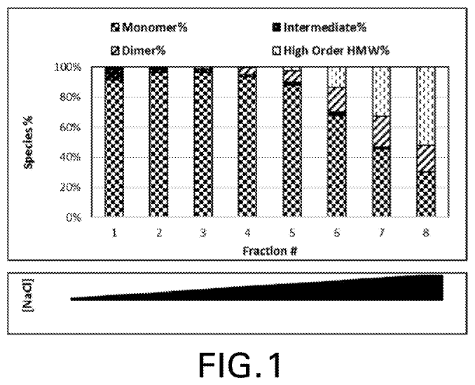

We first performed a salt gradient study using a salt concentration up to 300 mM for 15 CVs at pH 5.5. Fractions with UV between 100mAU ascending and 100mAU descending were collected for SEC analysis. While HMW1 increased with the increase of the salt concentration of the elution buffer, the HMW2 surprisingly decreased, with the highest HMW2 in the earlier fractions (FIG. 1). It appeared that the HMW2 had a weaker binding on the CEX column compared to the HMW1 and monomer species. Such atypical biophysical property posed a great challenge for CEX optimization to achieve a high monomer purity. By applying a step gradient for the elution, it was found that the HMW2 species was literally unchanged in the elution pool, while HMW1 species had been completely removed (FIG. 2). These observations suggested that the HMW2 species may have distinctive biophysical properties in terms of surface charge compared to HMW1 species. Therefore, characterization of these aggregates, especially the HMW2 species, becomes essential in understanding their behaviors including surface charge and hydrophobicity, which can help develop a process in removing aggregates more effectively. Meanwhile, exploration of using alternative resins was also attempted.

### 2. Study Using Other Resins

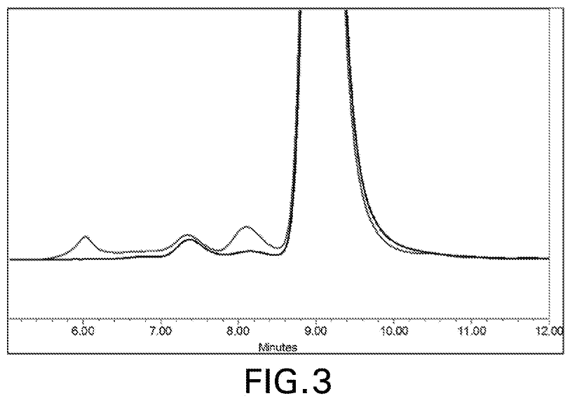

It seemed that high monomer purity could not be achieved solely by optimizing the CEX operation condition. Alternative approaches were sought by using different types of resins, such as hydrophobic interaction chromatographic (HIC) resins and anion and cation mixed-mode chromatographic (MMC) resins. However, the yield using HIC was very poor probably due to the very hydrophobic nature of the protein. The protein was tightly bound on the HIC column even under low salt or no-salt conditions, unless organic solvents such as ethanol were introduced (data not shown), which was not an ideal option for protein stability and product quality. Capto MMC offered some promising result by removing some level of the intermediate species, but not the dimer species [FIG. 3]. The HMW removal performance using difference resins were summarized in Table 1.

TABLE 1
Summary of HMW removal performance using different resins

| Chromatography | Resin | Mode | Results |
|---|---|---|---|
| HIC | Capto Phenyl | F/T | Poor recovery, |
| | | | poor separation |
| | Tosoh Butyl | F/T | Poor separation |
| | Phenyl Sepherose | F/T | Poor separation |
| MMC (CEX) | Capto MMC | B/E | Intermediate peak |
| | | | remained |
| MMC (AEX) | Capto Adhere ImPres | B/E | Intermediate peak |
| | | | remained |
| | MEP HyperCel | B/E | Intermediate peak |
| | | | Partially removed |
| | | | Dimer peak remained |
| CEX | FractoGel | B/E | Intermediate peak |
| | | | remained |
| | Poros XS | B/E | Intermediate peak |
| | | | remained |

### 3. Fractionation and Characterization of Aggregates

In order to develop a process to effectively remove all aggregates, a deep understanding of these aggregate species is needed. For this purpose, aggregate fractions were collected using a preparative SEC column from Tosoh with high purities for dimer, intermediate and monomer. The aggregates were then characterized to determine the main composition and their biophysical properties using SEC-MLAS, capillary electrophoresis, imaged capillary electrophoresis (iCE), and LC/MS. We also used analytical tools including CEX HPLC and HIC HPLC for relative surface charges and hydrophobicity. All characterization results were summarized in Table 2.

Fractionation

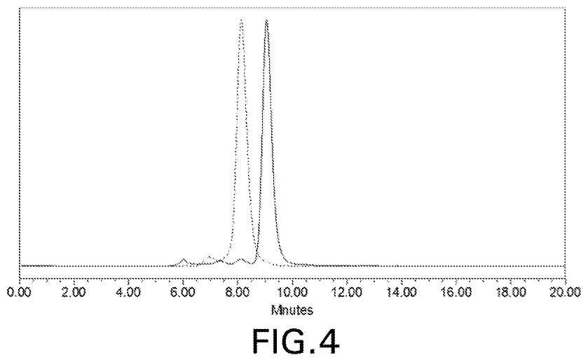

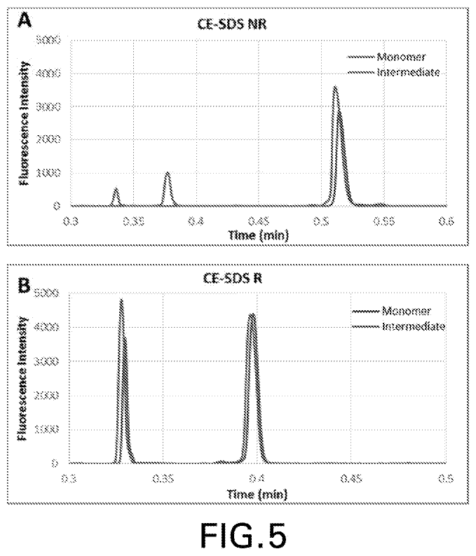

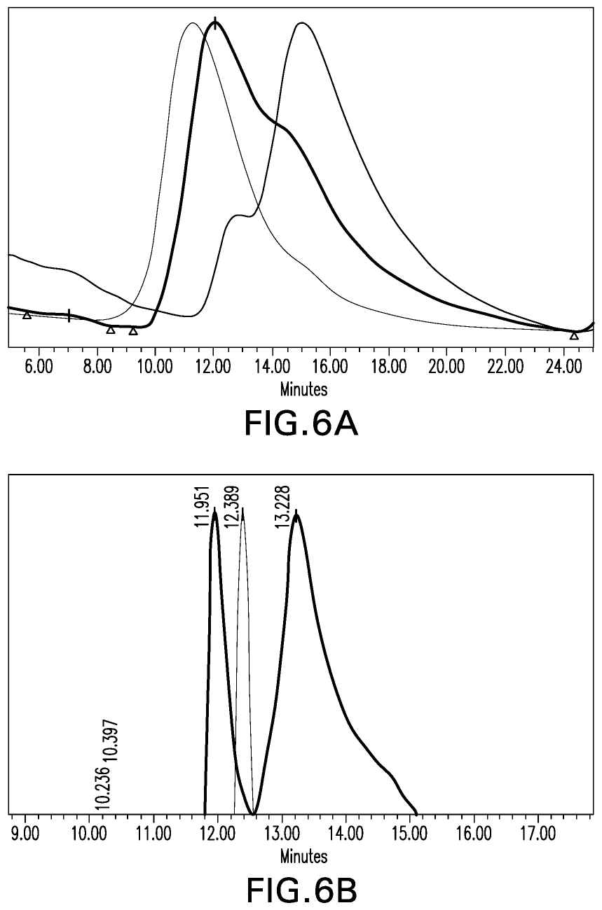

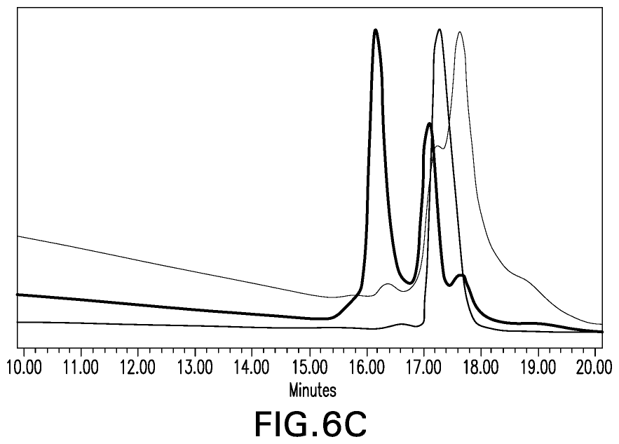

The fractions were collected and re-injected onto SEC to evaluate the purity. The SEC chromatogram of the purified intermediate species (>95% purity) was superposed with the starting material, as shown in FIG. 4.

Composition Analysis

The composition of the intermediate species was analyzed by using chip-based capillary electrophoresis and SEC-MALS. The LC/MS coupled with Fabricator digestion was also used to confirm the composition.

The electrophoregrams (R and NR) of the intermediate species and monomer were presented in Figures xx-xxy. Based on the reducing condition, the ratio of LC to HC was calculated to be ˜1.6, indicating higher level of overall LC than HC. On the other hand, two peaks with molecular weight of 30 and 54 kDa were shown in the intermediate species under non-reducing condition. Therefore, the 30 kDa peak and the 54 kDa peak were assigned as a single LC and covalently binding LC-LC, respectively. The intermediate species was composed of two main complexes: 1) a complex of a monomer non-covalently associated with a light chain; 2) a complex of a monomer non-covalently associated with a light chain dimer. The monomer/LL was the predominant intermediate species based on CE-NR result. Such composition assignment matched well with the molecular weight measurement of ˜200 kDa by SEC-MLAS.

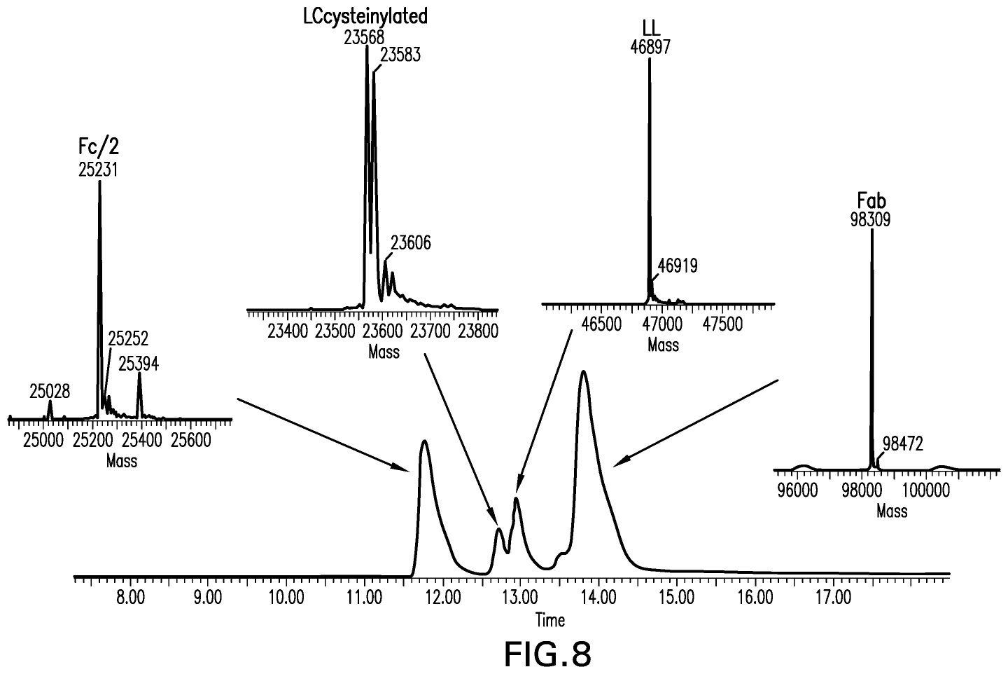

The composition was further confirmed using Fabricator digested LC/MS analysis. As shown in FIG. 8.

Biophysical Properties

We further investigated the biophysical properties of the intermediate species using imaged capillary electrophoresis for overall charge, analytical CEX for overall surface charge, and analytical HIC for overall surface hydrophobicity. Interestingly, the intermediate species was found to be not only less hydrophobic than the monomer, but also contains less surface charge. Such atypical biophysical behavior may be associated to their unique compositions.

$$LC \% = \frac{LC Area}{LC + HC} \times 100$$

$$HC \% = \frac{HC Area}{LC + HC} \times 100$$

Normalized ratio of LC to HC=(LC % in sample/LC % in Monomer)/(HC % in sample/HC % in Monomer)

Equations: calculation of LC/HC for the intermediate species

TABLE 2
Characteristics of aggregates for the anti-IP10 mAb

| | | | | Reduced CE | | | | | | | | |
|---|---|---|---|---|---|---|---|---|---|---|---|---|
| | | Purity | SEC-MALS | % | % | HC: | HC:LC | Non-reduced CE | | Hydrophobicity | Charge | Charge |
| | | % | MW (kDa) | HC | LC | LC | (normalized) | % Main | % Other | (normalized) | (pH5.5) | (pH7.0) |
| IP10 mAb | | | | | | | | | | | | |
| | Monomer | | 149 | 62.1 | 35.8 | | 1.0:1.0 | 98.9 | 1.1 | 100 | 100 | 100 |
| | Dimer | | 300 | 52.5 | 40.7 | | 1.2:1 | 65.1 | 34.9 | 102 | 133 | 106 |
| | Intermediate | | 200 | 50.0 | 45.9 | | 1.7:1 | 70.2 | 29.8 | 94 | 106 | 97 |

### Analytical CEX and HIC HPLC

The aggregates (Dimer and Intermediate) and monomer of the anti-IP10 mAb were injected onto an analytical CEX HPLC and an analytical HIC HPLC, respectively. For CEX, two running conditions (pH 5.5 and pH 7) were applied in order to evaluate the resolution between the dimer, intermediate and monomer. At pH 5.5, the dimer was very well separated from the monomer, but the intermediate species was co-eluted with monomer, which was exactly the case using CEX purification. However, when the running condition was adjusted to pH7, the intermediate species was eluted earlier, being separated from the monomer. Therefore, a strategy that includes using high pH wash to remove the intermediate species and using B/E to elute the monomer and retain the more strongly bound dimer on column becomes viable.

### Further Understanding of the Intermediate Species

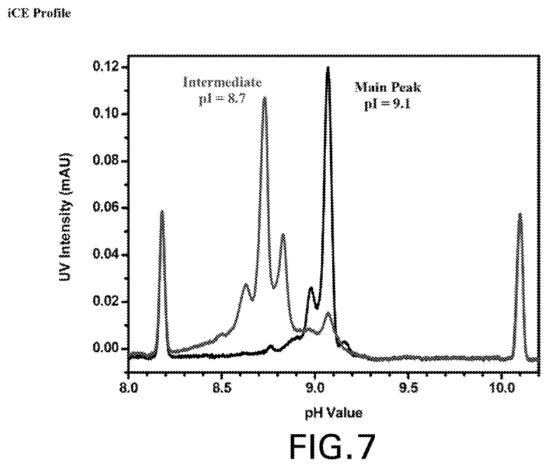

The intermediate species was further characterized by iCE, and LC/MS. As shown in FIG. 7, the intermediate species has a main pI value of 8.7, which is about 0.4 unit lower than the monomer.

### iCE Profile

The iCE profile for the intermediate species vs monomers (black line—monomers; red line—intermediate species) is shown in FIG. 7.

### ESI/Mass Spectrum

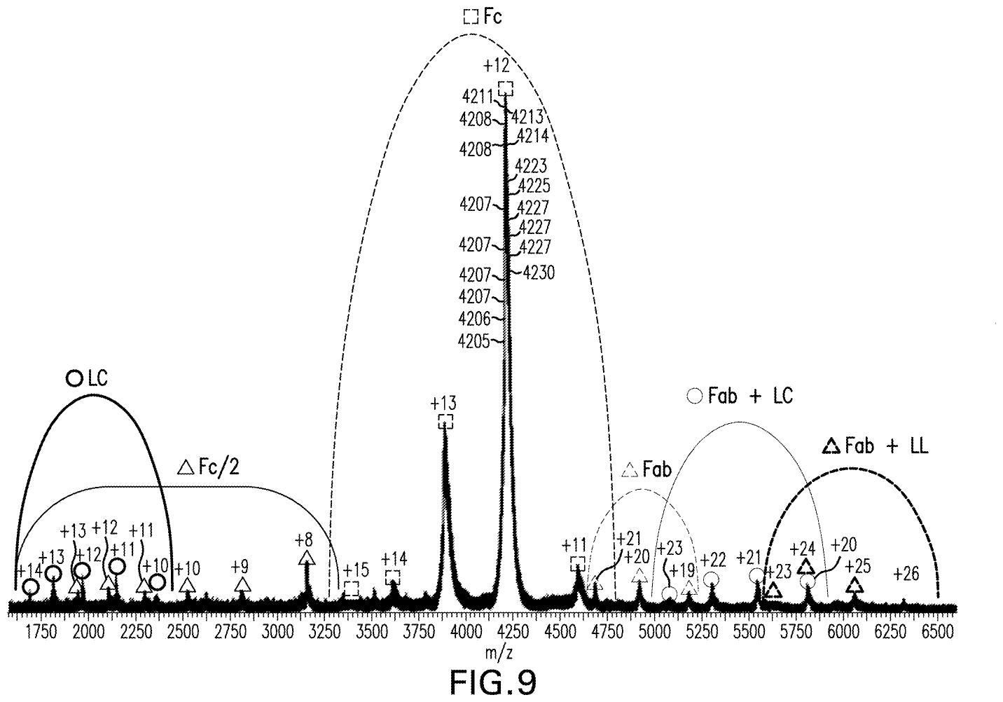

To further confirm the composition of the intermediate species, LC/MS analysis of Fabricator digestion combined with native nano ESI/MS was performed, as shown in FIGS. 8 and 9. Native ESI/MS combined with FabRICATOR digestion did not disturb non-covalent interactions. Non-covalently linked LC-Fab and LL-Fab were detected, indicating LC and LL dimer were bound to the Fab region.

### Process Optimization

### 1. pH Gradient

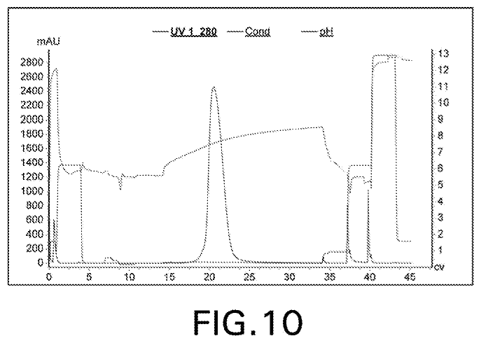

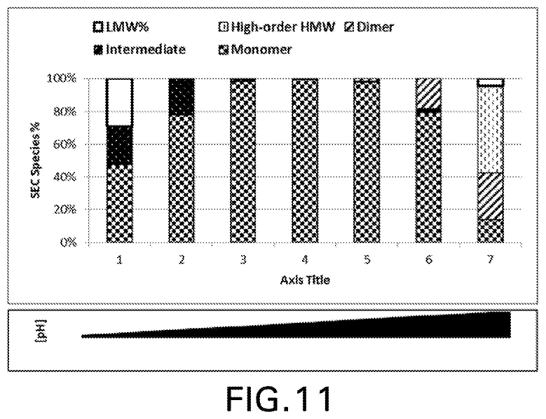

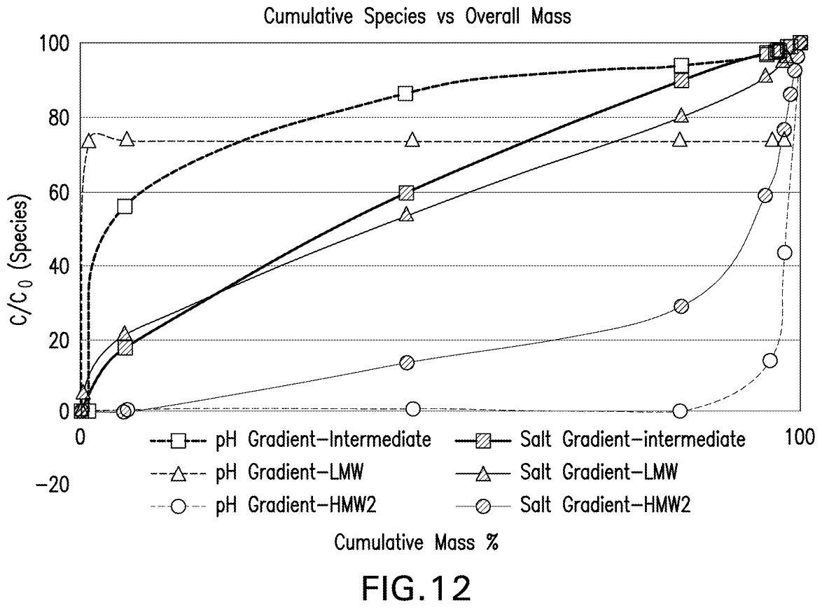

Since the intermediate species shows a lower pI value than the monomer, it is possible to remove the intermediate species by manipulating the working buffer pH. Therefore, a pH gradient using 30 mM phosphate buffer ranging from pH 5.5 to pH 8.5 was applied to the mAb protein A pool on the same CEX column, as shown in FIG. 10. Fractions were collected and subjected to SEC analysis. The species distribution from SEC for the linear pH gradient elution was illustrated in FIG. 11. Initially, more LMW and intermediate species were eluted earlier under a relatively lower pH condition, followed by monomer species. While elution pH increases, more dimer and high-order species starts eluting off the column. The results confirmed the findings from the analytical CEX HPLC that pH could be used to separate the intermediate species from monomer. Furthermore, the plot in FIG. 12 suggested that an operation window can be optimized to yield highly purity monomer pool. Therefore, a DOE study will be carried out to evaluate load amount, wash pH, wash salt concentration, and wash volume.

To further compare the selectivity toward separation of HMW between the salt gradient and pH gradient, the total cumulative content of aggregates percentage for each fraction (C/C₀) was plotted against the protein yield percentage. As we knew the intermediate species was eluted earlier than the monomer and the dimer species was eluted later, therefore, the plots with a sharper slope for the intermediate species and shallower slope for the dimer species would be the most ideal scenario, in which case, the intermediate species will be effectively washed out and the dimer species will be retained on the column.

The comparison between the salt gradient and pH gradient was plotted and shown in FIG. 12. Compared to the salt gradient, the pH gradient clearly provided superior separation between the intermediate, dimer, and monomer. With 10% protein yield, pH gradient provided >60% intermediate species clearance, versus just over 20% for the salt gradient. At mean time, >70% LMW was removed for the pH gradient versus 20% for the salt gradient. The dimer and high-order aggregate species were tightly bound on the column until pH or salt concentration reached certain levels. However, similar to the separation between intermediate species and monomer, a better separation between the dimer and monomer was rendered for the pH gradient. Overall, it seems that optimization the CEX pH condition is a better strategy in maximizing overall aggregate and LMW removal and minimizing product yield loss.

### 2. Combination of pH and Salt for Wash Buffer

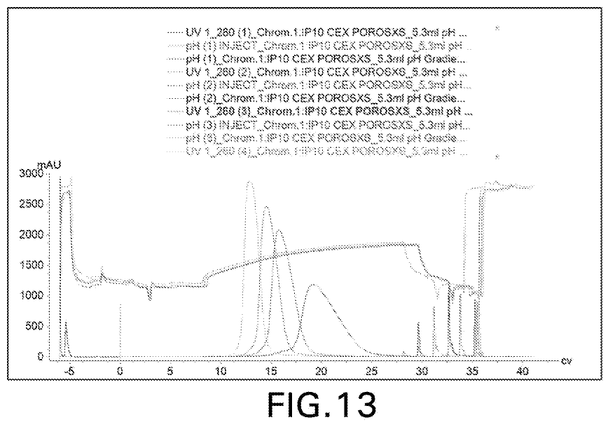

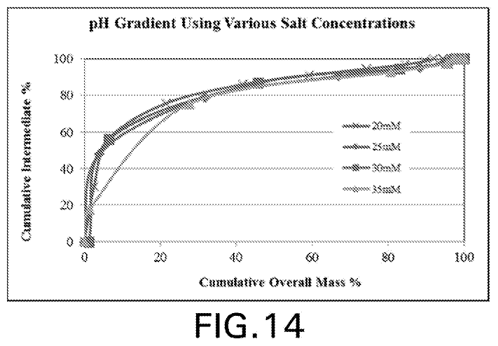

While the separation between the intermediate species and monomer was impacted by the operation pH, the conductivity of the running condition may still play an important role in the resolution as well as the product yield. Therefore, a serial study of pH gradient with various conductivities was performed and the intermediate species profiles were mapped out verse the overall mass for each condition, as shown in FIGS. 13 and 14.

### 3. Poros XS Using a High pH Wash in a Step Gradient

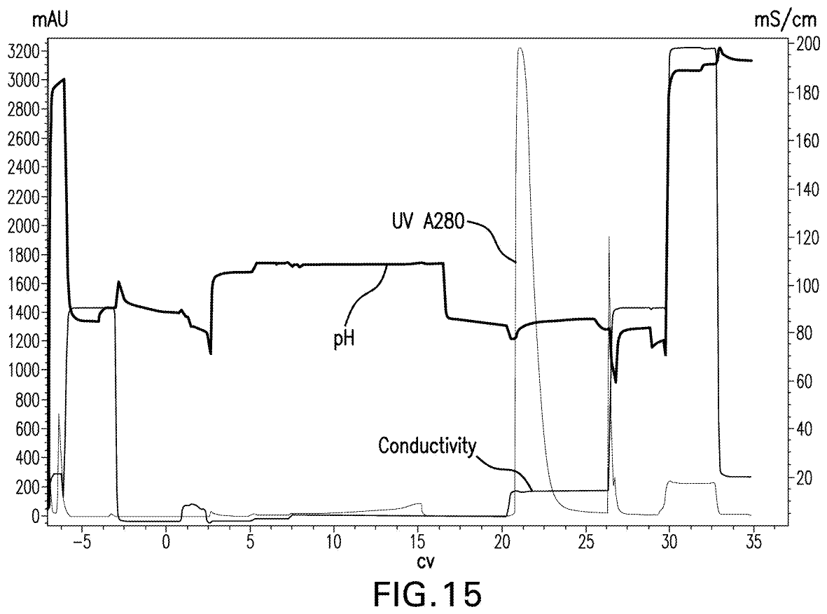

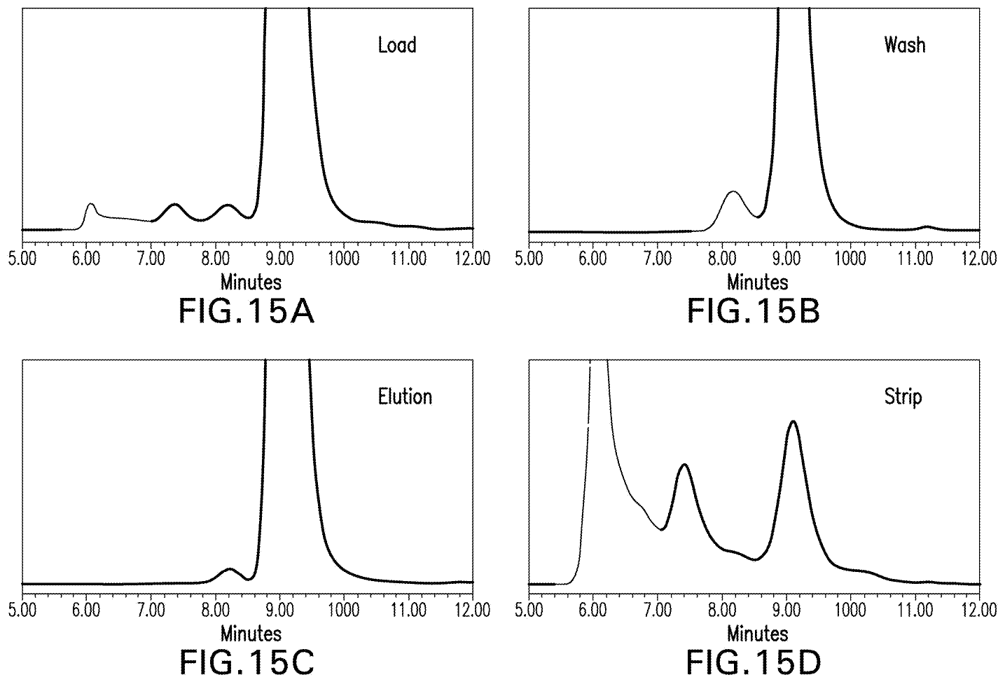

Excellent resolution between the intermediate and monomer has been achieved by using a pH gradient. However, a step gradient is preferred from manufacturing perspective. By evaluating the process parameters and taking the consideration of aggregate removal and product yield, an optimal condition (25 mM phosphate, pH 7.4) was chosen to evaluate the pH step gradient. The CEX chromatogram and respective SEC profiles (A—Load, B—CEX high pH wash, C—CEX elution, and D—CEX strip) were shown in FIG. 15.

### 4. CEX DOE Study

Our initial study on column using different pH and salt concentrations in the wash buffer provided us a range where the optimal condition might be. We determined that the best range for wash pH was 7.2-7.6, and the range for wash salt [NaCl] was 24-30 mM over 3-7 CVs. The loading amount (20-30 mg protein/mL resin) was also included as a study factor.

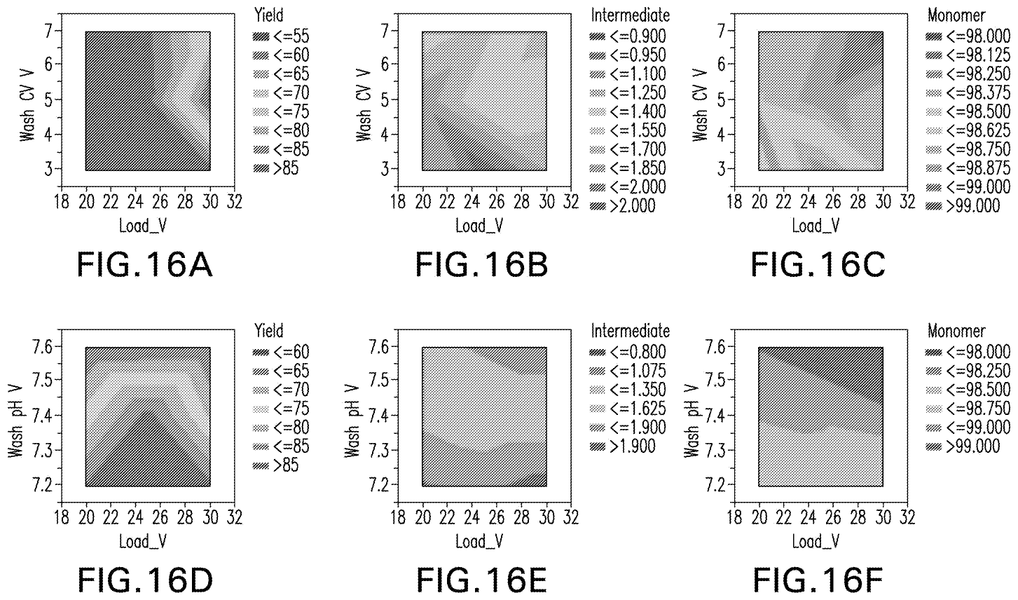

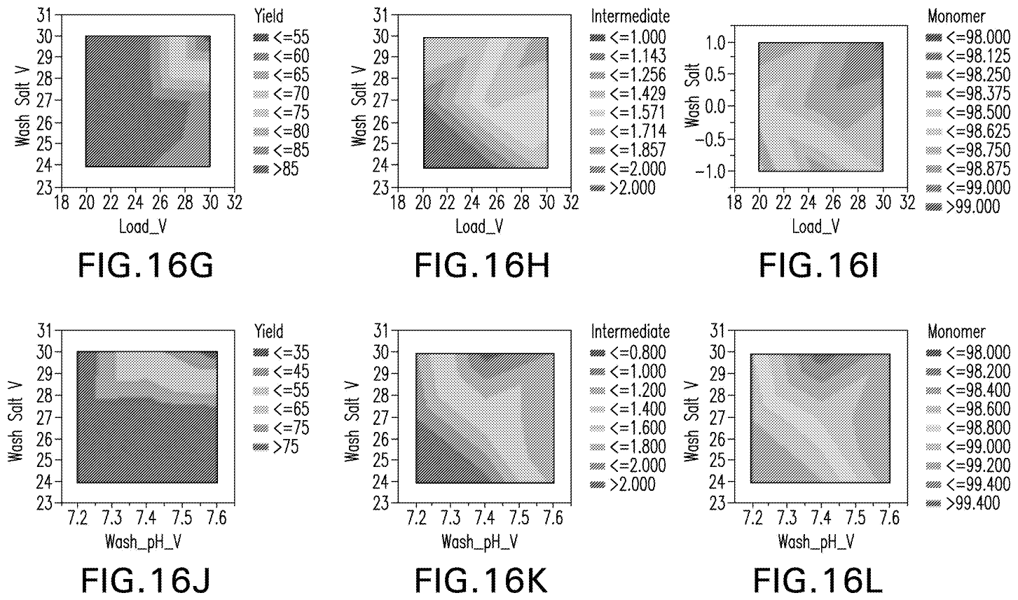

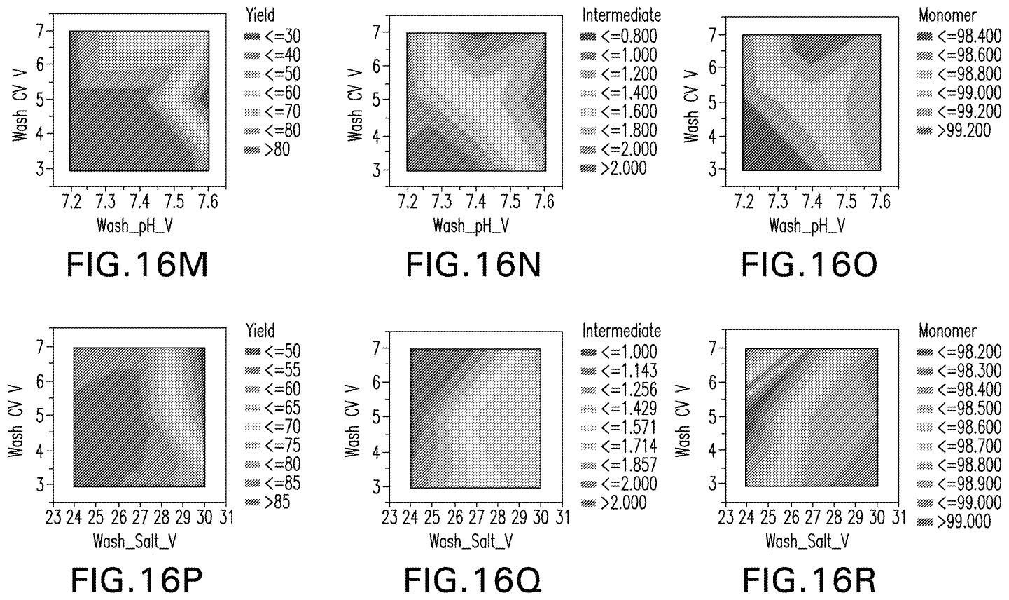

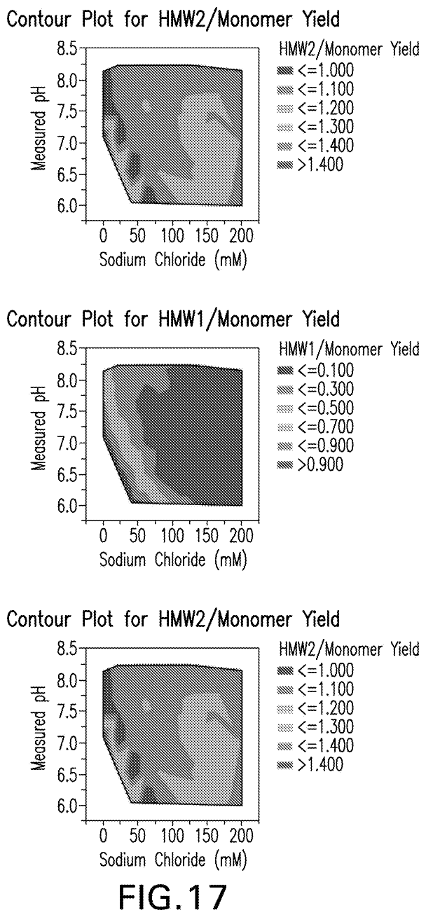

A DOE experimental design was used to characterize the CEX process. This study was important to define a design space and operation range for good product quality and robust process. We focused the study on the wash step in terms of aggregate clearance and product yield. The loading material was a typical Protein A pool from an un-optimized Protein A condition. Both dimer and intermediate were present in the Protein A, with each more than 2.5%. For this study, an Omnifit column with a 5 mL Poros XS resin was used. For this experiment, a custom design composing of 18 runs was generated using JMP10.0. Four factors with 3 levels were included in the experiment, namely load amount (20, 25, and 30 mg/mL resin), wash pH (7.2, 7.4, and 7.6), wash salt [NaCl] (24 mM, 27 mM, and 30 mM), and wash volume (3CVs, 5CVs, and 7CVs). The elution samples were tested for intermediate species and monomer by SEC, recovery by A280 spectrophotometer, HCP by ELISA, and DNA by qPCR. The contour plots for the SEC and monomer recovery data are shown in FIG. 16.

The % monomer, % intermediate, and recovery data were modeled in IMP10. Since residual HCP and DNA were close to, or below, the detection limit of the assay—and thus acceptable in all instances—for the mAb recovered from the Poros XS under all process conditions, these results were not included in the model. Modeling of all three responses together resulted in a good, statistically significant, model (p<0.05). Results from the process model indicated that wash pH had the greatest effect on % intermediate, % monomer and % recovery.

The yield was mostly affected by wash pH and wash salt concentration. The load and wash CV were found not to have profound effect on the yield.

The strong correlation between the intermediate species and wash pH was observed.

### Discussion

We found the intermediate species was more acidic than the monomer, which prompted us to explore the surface charge modification by pH modulation instead of salt addition. A CEX polishing step using a high pH wash to remove the intermediate species effectively was developed. We successfully implemented a platform purification including Protein A (ProA)→CEX→AEX to control process impurities (HCP, DNA, residual leached Protein A) and aggregates to achieve CEX elution pool with >99% monomer purity and >80% yield.

We identified the intermediate species was composed of two main complexes: a monomer non-covalently associated with either a light chain or a light chain dimer. Although the mAbs containing a third light chain have been reported and characterized, the mAb containing a light chain dimer with such high percentage has not been reported. Interestingly, even though both complexes showed as one single intermediate peak on SEC, they appeared to have slightly different surface charge since our high pH wash buffer was more effective in removing the complex containing light-chain dimer. The question now is how to explain 1) the intermediate species contains less surface charge than the monomer; 2) the complex of monomer with the light chain dimer was less charged than the one with a single light chain. To answer it, we sought to learn the electrostatics of the intermediate species under different pH environments. We used APBS (Adaptive Poisson-Boltzmann Solver) to determine the overall surface charges of the two intermediate species and monomer under pH 5.5 and 7.4.

The unique biophysical property of the intermediate species was also reflected in its unusual hydrophobicity relative to monomer. We used a HPLC hydrophobic interaction chromatography under the bind elution mode to evaluate the hydrophobicity of each species. Interestingly, the intermediate species was eluted earlier than the monomer, suggesting the intermediate species was less hydrophobic than the monomer. The decreasing hydrophobicity of the intermediate species might be due to the fact that certain hydrophobic patches may be buried inside caused by the association between the mAb monomer and LC or LL. Equally out of the ordinary, during our ProA chromatographic step development, we found that the intermediate species was eluted earlier on the affinity ProA column compared to the monomer species (data not shown). Such phenomena might be explained 1) ProA interactions mainly consist of hydrophobic interactions as well as some hydrogen bonding and two salt bridges; 2) Although the binding site between the mAb and LC/LL was found to be Fab region of the monomer, some research work has confirmed that there was a significant structural coupling between the Fab arms and Fc and the content of the Fab can have an impact on Fc binding to various receptors; 3) although the extra LC (s) was associated to the mAb Fab, possible steric hindrance might cause weaker binding between the intermediate species and protein A resin.

### CONCLUSION

Size exclusion high performance liquid chromatography analysis of a human monoclonal antibody (mAb) showed the presence of a new aggregate species between the dimer and monomer species. However, extensive characterization of this species, referred to as “intermediate”, revealed that the intermediate species is a different kind in terms of size and biophysical attributes. The intermediate was determined to be mainly a complex containing a mAb associated with two extra light chains with a molecular weight of 200 kDa. The covalently bound light chain dimer was found to be non-covalently associated with the Fab portion of the monomer.

In most cases, aggregates are found to be more hydrophobic and more positively charged compared to its monomer species. A simple binding and elution on CEX can be easily implemented to achieve HMW clearance <1% in the elution pool. However, the intermediate aggregate in this anti-IP10 mAb was found not only to be less hydrophobic than the monomer but also has slightly less surface charge than the monomer, which presented a big challenge for downstream purification process. HIC is not a desirable polishing step in the mAb purification due to following challenges: a) extremely high hydrophobicity makes HIC bind/elution or flow through mode hard to implement; b) weaker binding to the column due to its lower hydrophobicity of the intermediate aggregate makes HIC flow through unsuitable. Therefore, a suitable polishing strategy is needed to remove aggregates more effectively.

It appears that neither HIC chromatography nor CEX chromatography was suitable for the intermediate aggregate removal for this mAb. However, by manipulating the CEX wash buffer pH, it was found that the intermediate species that has approximate MW of 200 kDa became less charged than the monomer, which makes it bind on the CEX column weaker than the monomer, thus enabling the separation between the two species. With the focus on the wash strategy, we developed a polishing process using Poros XS resin that is capable of removing the intermediate aggregate with an effective wash and removing all other aggregate species by optimizing the elution buffer. For this mAb, a two-column process including protein A and CEX was sufficient in impurity removal as well as HMW removal. We believe that such high pH wash strategy can be applicable to the situation where aggregates show lower pI values than monomer.

Moreover, it was found that the intermediate species in this mAb was more weakly bound onto the protein A column (data not shown), which may enable us to use the capture step to remove certain level of intermediate species by implementing an effective wash or peak cutting strategy. Additionally, we evaluated using Mercapto-Ethyl-Pyridine (MEP) hydrophobic charge induction resin to remove aggregate, the intermediate aggregate in particular. Such work will be presented in separate papers.

### REFERENCES

1. S K Singh, N Afonina, M Awwad, et, J Pharm Sci 2010; 99: 3302-21; An industry perspective on the monitoring of subvisible particles as a quality attribute for protein therapeutics
2. C J Roberts, Protein aggregation and its impact on product quality. Curr. Opin Biotechnol 2014; 30C: 211-7
3. A S Rosenberg, Effects of protein aggregates: an immunologic perspective. Asps J 2006; 8:E501-7
4. Ratanji K D, et al. “Immunogenicity of therapeutic proteins: influence of aggregation” J Immunotoxicol. 2014, 11(2):99-109
5. Maria Vazquez-Rey, Dietmar Lang, Aggregates in Monoclonal antibody manufacturing processes, B&B, 2011; 108, 1494-1508
6. Cromwell M E, Hilario E, Jacobson F. Protein aggregation and bioprocessing. Aaps J 2006; 8:E572-9
7. H. Liu, J. Ma, Charles Winter, Robert Bayer, Recovery and Purification process development for monoclonal antibody production, mAbs 2:5 (2010) 480-499
8. Kelley B, Blank G, Lee A. Downstream Processing of Monoclonal Antibodies: Current Practices and Future Opportunities. In: Process Sacle Purification of Antibodies. Gottschalk U, Editor. John Wiley & Sons, Inc 2009; 1-23
9. Shukla A A, Hubbard B, Tressel T, Guhan S, Low D. Downstream processing of monoclonal antibodies Application of platform approaches. J Chromatogr B 2007; 848:28-39
10. W. Marek, R. Muca, S. Wo's, W. Pi, atkowski, D. Antos, Isolation of monoclonal antibody from a Chinese hamster ovary supernatant. I: Assessment of different separation concepts, J. Chromatogr. A 1305 (2013) 55-63
11. W. Marek, R. Muca, S. Wo's, W. Pi, atkowski, D. Antos, Isolation of monoclonal antibody from a Chinese hamster ovary supernatant. II: Dynamics of the integrated separation on ion exchange and hydrophobic interaction chromatography media, J. Chromatogr. A 1305 (2013) 64-75
12. Z. Xu, J. Li, J. X. Zhou, Process development for robust removal of aggregates using cation exchange chromatography in monoclonal antibody purification with implementation of quality by design, Prep. Biochem. Biotechnol. 42 (2012) 183-202
13. Natalia Gomez, Abigail Vinson, et, Biotech and Bioeng. Vol 105, No 4, Mar. 1, 2010, 748-760, Triple light chain antibodies: factors that influence its formation in cell culture
14. Natalia Gomez, Jun Ouyang, Mary Nguen, et, Biotechnol. Prog., 2010, Vol. 26, No5, Effect of temperature, pH, Dissolved Oxygen, and hydrolysate on the formation of triple light chain antibodies in cell culture
15. Rachael Wollacott, Paul Casaz et, mAbs 5:6, 925-935, 2013, Analytical characterization of monoclonal antibody therapeutic reveals a three-light chain species that is efficiently removed using hydrophobic interaction chromatography
16. Connie Lu, Dandan Liu et, mAbs 5:1, 102-113, 2013, Characterization of monoclonal antibody size variants containing extra light chains
17. Jie Chen, Jen Tetrault, et, JChromA, 1217 (2010) 216-224, The distinctive separations attributes of mixed-mode resins and their application in monoclonal antibody downstream purification process
18. Dong Gao, Li-Li Wang, Dong-Qiang Lin, Shan-Jing Yao, Evaluating antibody monomer separation from associated aggregates using mixed-mode chromatography, J. Chromatogr A, 1294 (2013) 70-75
19. Brian Kelley, High-Throughput Screening of Chromatographic Separations: IV. Ion-Exchange, B&B, Vol 100, No 5, Aug. 1, 2008, 950-963
20. Sanchayita Ghose, Yingying Tao, et, mAbs 5:5, 795-800, 2013, Purification of monoclonal antibodies by hydrophobic interaction chromatography under no-salt conditions
21. Dell Farnan and G. Tony Moreno, Anal Chem, 2009, 81, 8846-8857, Multiproduct High-resolution Monocloneal antibody charge variant separations by pH gradient Ion-exchange chromatography
22. Timothy M. Pabst, Dorota Antos, Giorgio Carta, Natarajan Ramasubramanyan, Alan K. Hunter, JChromA, 1181 (2008) 83-94, Protein separations with induced pH gradients using cation-exchange chromatographic columns containing weak acid groups
23. Tangir Ahamed, Beckley K. Nfor, Peter Verhaert, Gijs van Dedem, et, JChromA, 1164(2007) 181-188, pH-gradient ion-exchange chromatography: An analytical tool for design and optimization of protein separations
24. Paul Ng, Jie He, Mark Snyder, JChromA 1216(2009) 1372-1376, Separation of protein mixtures using pH-gradient cation-exchange chromatography
25. Hennifer Rea, Tony Moreno, Yun Lou, Dell Farnan, JPharmBiomedAnal 54 (2011)317-323, validation of a pH gradient-based ion-exchange chromatography method for high-resolution monoclonal antibody charge variant separations
26. Tangir Ahamed, Sreekanth Chilamkurthi, et, J Chrom. A, 1194(2008) 22-29, Selection of pH-related parameters in ion-exchange chromatography using pH-gradient operations
27. Frieder Kroner, Jurgen Hubbuch, JChromA 1285 (2013) 78-87, Systematic generation of buffer systems for pH gradient ion exchange chromatography and their application
28. Frieder Kroner, Alexander Hanke, et, J ChromA, 1311 (2013) 55-64, Analytical characterization of complex, biotechnological feedstocks by pH gradient ion exchange chromatography for purification process development
29. Jack Kramarzyk, Brian Kelley, Jonathan Coffman, B&B, 100 (2008) 707-720, High-Throughput Screening of Chromatographic separations: II. Hydrophobic Interaction
30. Jie Chen, Jenifer Tetrault, Arthur Ley, J Chrom A, 1177(2208) 272-281, Comparison of standard and new generation hydrophobic interaction chromatography resins in the monoclonal antibody purification process
31. Agnes Voitl a, Thomas Müller-Spätha, b, Massimo Morbidelli, J Chrom A, 1217(2010), 5753-5760, Application of mixed mode resins for the purification of antibodies
32. Jerome Pezzinia, b, Gilles Jouclaa, René Gantierb, Magali Toueille, J Chrom A, 1218 (2011) 8197-8202, Antibody capture by mixed-mode chromatography: A comprehensive study from determination of optimal purification conditions to identification of contaminating host cell proteins
33. Vijesh Kumar, Anurag S. Rathore, J Chrom A, 1368 (2014) 155-162, Two-stage chromatographic separation of aggregates for monoclonal antibody therapeutics

### EQUIVALENTS

Those skilled in the art will recognize or be able to ascertain using no more than routine experimentation, many equivalents of the specific embodiments of the invention described herein. Such equivalents are intended to be encompassed by the following claims.

### INCORPORATION BY REFERENCE

All patents, pending patent applications, and other publications cited herein are hereby incorporated by reference in their entireties.

1. A method of purifying a monomeric monoclonal antibody from a mixture which comprises the monomeric monoclonal antibody and one or more contaminants, comprising:
a) subjecting the mixture to cation exchange chromatography (CEX) matrix, wherein the monomeric monoclonal antibody binds to the CEX matrix;
b) contacting the CEX matrix with a wash solution at a pH which is between about 7 and about 7.8; and
c) eluting the monomeric monoclonal antibody from the CEX matrix into an elution solution, thereby purifying the monomeric monoclonal antibody.

2. The method of claim 1, wherein the contaminants are selected from aggregates of the monoclonal antibody, host cell proteins, host cell metabolites, host cell constitutive proteins, nucleic acids, endotoxins, viruses, product related contaminants, lipids, media additives and media derivatives.

3. The method of claim 1, wherein aggregates of the monoclonal antibody comprise dimers, multimers, and an intermediate aggregate species.

4. The method of claim 1, wherein the mixture has been obtained by an affinity chromatography.

5. The method of claim 1, wherein the elution solution is not subjected to a second chromatography step.

6. The method of claim 1, wherein the elution solution is further subjected to a second chromatography step.

7. The method of claim 6, wherein the second chromatography is selected from an ion exchange chromatography, a hydrophobic interaction chromatography, and a mix-mode chromatography.

8. The method of claim 1, wherein the pH of the wash solution is between about 7.2 and about 7.6.

9. The method of claim 1, wherein the salt concentration of the wash buffer is between about 20 and 40 mM.

10. The method of claim 1, wherein the salt concentration of the wash buffer is between about 24 and 30 mM.

11. The method of claim 1, wherein the intermediate aggregate species is removed in step (b).

12. The method of claim 1, wherein the mixture is selected from a harvested cell culture fluid, a cell culture supernatant, and a conditioned cell culture supernatant, a cell lysate, and a clarified bulk.

13. The method of claim 12, wherein the cell culture is a mammalian cell culture.

14. The method of claim 13, wherein the cell culture is a Chinese Hamster Ovary (CHO) cell culture.

15. The method of claim 1, wherein the monoclonal antibody is an anti-IP10 antibody.

16. The method of claim 15, wherein the anti-IP10 monoclonal antibody comprises heavy chain CDR1, CDR2, and CDR3 sequences of SEQ ID NOs: 1, 2, and 3, and light chain CDR1, CDR2, and CDR3 sequences of SEQ ID NOs: 6, 7, and 8, respectively.

17. The method of claim 15, wherein the anti-IP10 monoclonal antibody comprises a heavy variable region sequence and a light chain variable region sequence of SEQ ID NOs: 4 and 9, respectively.

18. The method of claim 15, wherein the anti-IP10 monoclonal antibody comprises the full-length heavy chain amino acid sequence and the full-length light chain amino acid sequence of SEQ ID NOs: 5 and 10, respectively.

19. The method of claim 1, wherein the monomeric monoclonal antibody is purified to at least 90% monomer purity.

20. The method of claim 1, wherein the monomeric monoclonal antibody is purified to at least 95% monomer purity.

21. (canceled)
